(19) United States
(12) Patent Application Publication
(10) Pub. No.: US 2011/0088129 A1
(43) Pub. Date: Apr. 14, 2011

(54) Bollworm Insect Resistance Management in Transgenic Plants

(76) Inventors: Carmen Sara Hernandez, Burjassot (Valencia) (ES); Adri Van Vliet, Gent (BE); Jeroen Van Rie, Eeklo (BE); Juan Ferre Manzanero, Burjassot (valencia) (ES)

(21) Appl. No.: 12/997,133

(22) PCT Filed: Apr. 16, 2009
(86) PCT No.: PCT/EP2009/002788

§ 371 Date: Dec. 9, 2010

Related U.S. Application Data
(63) Continuation-in-part of application No. 12/214,022, filed on Jun. 16, 2008.

(30) Foreign Application Priority Data
Jun. 13, 2008 (EP) 08010791.5
Oct. 21, 2008 (NZ) 572189

(51) Int. Cl. A01H 5/00; A01N 37/18; A01P 7/04; G06Q 99/00; A01C 7/00

(52) U.S. Cl. 800/302; 514/4.5; 705/317; 111/200

(57) ABSTRACT

This invention relates to the use of a combination of different proteins insecticidal to Helicoverpa zeaor Helicoverpa armigerain an insect resistance management process, wherein such proteins are: a) a Cry2A protein such as Cry2Aa, Cry2Ab, or Cry2Ae and b) a Cry1A, Cry1F or VIP3A protein, particularly wherein such proteins binds saturably to the insect midgut membrane of Helicoverpa zeaor Helicoverpa armigera, as well as plants and seeds expressing such combination of proteins, which are used to delay or prevent the development of resistance in populations of such insect species.

### FIELD OF THE INVENTION

The present invention relates to the field of plant pest control, particularly insect control. This invention relates to the use of transgenic plant cells and plants in an insect resistance management process, wherein the genomes of said cells and plants (or more typically, predecessor plant cells or plants) have been provided with at least two genes, each encoding a different protein insecticidal to Helicoverpa zea or Helicoverpa armigera, wherein such proteins bind saturably to the brush border membrane of such insect species, which proteins are: a) a Cry2A protein and b) a Cry1A, Cry1F or VIP3A protein, such as a VIP3A, a Cry1Ac, a Cry1Ab or a Cry1A.105 protein. In one embodiment, such plants are used to delay or prevent the development of resistance to crop plants in populations of the cotton bollworm.

Also, in the present invention the simultaneous or sequential use of a Cry2A protein and a VIP3A, Cry1A or Cry1F protein or plants expressing such Cry2A protein and a VIP3A, Cry1A or Cry1F protein, to delay or prevent resistance development in cotton bollworms, particularly Helicoverpa zea or Helicoverpa armigera, is provided.

Such transformed plants have advantages over plants transformed with a single insecticidal protein gene, or plants transformed with a Cry1F- and a Cry1A-encoding gene, especially with respect to the delay or prevention of resistance development in populations of cotton bollworms, against the insecticidal proteins expressed in such plants.

This invention also relates to a process for the production of transgenic plants, particularly corn, cotton, rice, soybean, sorghum, tomato, sunflower and sugarcane, comprising at least two different insecticidal Cry proteins that show no competition for binding to the binding sites in the midgut brush border of Helicoverpa zea or Helicoverpa armigera larvae. Simultaneous expression in plants of chimeric genes encoding a Cry2A protein and a VIP3A, Cry1F or Cry1A protein, particularly a VIP3Aa, Cry1Ab or Cry1Ac protein, is particularly useful to prevent or delay resistance development of populations of cotton bollworms against the insecticidal proteins expressed in such plants.

This invention further relates to a process for preventing or delaying the development of resistance in populations of Helicoverpa zea or Helicoverpa armigera to transgenic plants expressing a VIP3 or a Cry1A and/or a Cry1F protein, comprising providing such plants also with a gene expressing a Cry2A protein. Since such Cry2A protein and such Cry1A or VIP3 or Cry1F protein do not compete for specific binding sites in the midgut brush border of Helicoverpa zea or Helicoverpa armigera larvae, these combinations are useful for securing long-lasting protection against said larvae.

This invention also relates to a method to control Helicoverpa zea or Helicoverpa armigera insects in a region where populations of said insect species have become resistant to plants comprising a VIP3, Cry1F and/or a Cry1A protein, comprising the step of sowing, planting or growing in said region, seeds or plants containing at least a gene encoding a Cry2A protein. In one embodiment of the invention, said plants can also comprise (besides the gene encoding a Cry2A protein) a gene encoding another insecticidal protein which does not share binding sites with such Cry2A, VIP3, Cry1F or Cry1A protein in Helicoverpa zea or Helicoverpa armigera.

### BACKGROUND OF THE INVENTION

Insect pests cause huge economic losses worldwide in crop production, and farmers face every year the threat of yield losses due to insect infestation. Genetic engineering of insect resistance in agricultural crops has been an attractive approach to reduce costs associated with crop-management and chemical control practices. The first generation of insect resistant crops have been introduced into the market since 1996, based on the expression in plants of insecticidal proteins derived from the gram-positive soil bacterium Bacillus thuringiensis (abbreviated herein as “Bt”).

In contrast to the rapid development of insect resistance to some synthetic insecticides, so far insect resistance to plant-incorporated insecticidal proteins such as B. thuringiensis proteins has not been reported despite many years of use. This may be because of the insect resistance management programs which are being used for such transgenic plants, such as the expression of a high dose level of protein for the main target insect(s), and the use of refuge areas (either naturally present or structured refuges) containing plants without such insecticidal proteins. Procedures for expressing B. thuringiensis or other insecticidal protein genes in plants in order to render the plants insect-resistant are well known in the art and provide a new approach to insect control in agriculture which is at the same time safe, environmentally attractive and cost-effective. An important determinant for the continued success of this approach will be whether (or when) insects will be able to develop resistance to insecticidal proteins expressed in transgenic plants. In contrast to a foliar application, after which insecticidal proteins are typically rapidly degraded, the transgenic plants will exert a continuous selection pressure on the insects. It is clear from laboratory selection experiments that a continuous selection pressure can lead to adaptation to insecticidal proteins, such as the B. thuringiensis Cry proteins, in insects.

Helicoverpa zea and Helicoverpa armigera are amongst the most significant polyphagous lepidopteran pest species in the New and Old World, respectively. These insects have a history of rather rapid resistance development to insecticides, and they are typically less sensitive to many Bt-derived insecticidal proteins compared to important other lepidopteran insect pests. Hence these insect species are amongst the most likely candidates to develop resistance to Bt-plants, such as Bt cotton or Bt corn plants.

The most widely used proteins introduced in plants for control of Lepidopteran insects include the Cry1A, Cry1F and VIP3A proteins. Based on competition binding assays, it has been proposed that a Cry1F protein competes for the same midgut binding site as Cry1Ac in Helicoverpa zea and Helicoverpa armigera. Moreover, no evidence was found for any unshared sites for Cry1F in these insects species (Hernandez and Ferre, 2005). Hence a combination of these two proteins in the same plant is not a suitable approach for resistance management of Helicoverpa zea or Helicoverpa armigera insects. Only a low affinity of Cry1Fa for the Cry1Ac binding site was found, this low affinity likely reflects the low toxicity observed for the Cry1F protein in these insect species (Liao et al., 2002).

There appears to be a generally accepted proposition that the mode of action of Cry2 toxins is unique, and different from other three-domain Cry toxins, due to their non-specific and/or non-saturable binding to an unlimited number of binding sites (English et al., 1994; Lee et al., 2006). Since the publication by English et al. (1994), the binding characteristics of the Cry2A protein described therein have apparently been reiterated, and several authors still refer to the method described therein for the preparation of Cry2A protein for binding assays (e.g., Luo et al., 2007). Also, EPA biopesticide factsheet 006487 (2002) states that the Cry2Ab protein, and Cry2 proteins in general, produce highly potent ion channels to compensate for binding either to themselves or to a large collection of non-specific binding sites. (www.epa.gov/opp00001/biopesticides/ingredients/factsheets/factsheet_{—}006487.htm)

Also, English et al. (1994) and Karim et al. (2000b) reported at least partial competition or the sharing of a common binding site for a Cry1A and Cry2A protein in Helicoverpa zea. Also, USDA-APHIS petition for non-regulated status 06-298-01p (2006) states that a Cry1A and Cry2A protein share many common binding sites (www.aphis.usda.gov/brs/aphisdocs/06_{—}29801p.pdf).

There is no report available that demonstrates saturable binding of a Cry2A protein based on a direct saturability assay, wherein a fixed concentration of binding sites (i.e., BBMVs) are used to which increasing concentrations of labeled protein are added. In contrast to the reports and findings in the art, the inventors conclusively show herein that a Cry2A toxin can bind in a specific and saturable manner to receptors in susceptible insects, and that a Cry2A toxin does not share (or compete for) binding sites with a Cry1A toxin in the cotton bollworms, H. zea and H. armigera.

The current document contains the first report showing that Cry2A proteins bind saturably to the midgut brush border membrane of susceptible insects in a direct saturability assay, and also contains the first report analyzing binding competition between different Cry2A proteins.

### SUMMARY OF THE INVENTION

Provided herein is a method of controlling Helicoverpa zea or Helicoverpa armigera infestation in transgenic plants while securing a slower buildup of Helicoverpa zea or Helicoverpa armigera insect resistance development to said plants, comprising expressing a combination of a) a Cry2Ae protein insecticidal to said insect species and b) a Cry1A, Cry1F or VIP3A protein insecticidal to said insect species, in said plants.

Also provided herein is a method for preventing or delaying insect resistance development in populations of the insect species Helicoverpa zea or Helicoverpa armigera to transgenic plants expressing insecticidal proteins to control said insect pest, comprising expressing a Cry2Ae protein insecticidal to Helicoverpa zea or Helicoverpa armigera in combination with a Cry1A, Cry1F of VIP3A protein insecticidal to Helicoverpa zea or Helicoverpa armigera in said plants.

In one embodiment of this invention, a method is provided to control Helicoverpa zea or Helicoverpa armigera in a region where populations of said insect species have become resistant to plants expressing a VIP3A, Cry1A or a Cry1F protein, comprising the step of sowing or planting in said region, plants expressing at least a Cry2Ae protein insecticidal to Helicoverpa zea or Helicoverpa armigera.

Further provided herein is a method to control Helicoverpa zea or Helicoverpa armigera in a region where populations of said insect have become resistant to plants expressing a Cry2Ae protein, comprising the step of sowing or planting in said region, plants expressing a Cry1F, VIP3, or Cry1A protein insecticidal to Helicoverpa zea or Helicoverpa armigera.

Also provided in accordance with this invention is a method for obtaining plants comprising chimeric genes encoding at least two different insecticidal proteins, wherein said proteins do not share binding sites in larvae of the species Helicoverpa zea or Helicoverpa armigera as determined in competition binding experiments using brush border membrane vesicles of said insect larvae, comprising the step of obtaining plants comprising a plant-expressible chimeric gene encoding a Cry2Ae protein insecticidal to Helicoverpa zea or Helicoverpa armigera and a plant-expressible chimeric gene encoding a Cry1A, VIP3 or Cry1F protein insecticidal to Helicoverpa zea or Helicoverpa armigera. Further provided herein is such method wherein said plants are obtained by transformation of a plant with plant-expressible chimeric genes encoding said Cry2Ae and Cry1A, VIP3 of Cry1F proteins, and by obtaining progeny plants and seeds of said plants comprising said chimeric genes; or by the crossing of a parent plant comprising said Cry2Ae-encoding chimeric gene with a parent plant comprising said Cry1A-, VIP3- or Cry1F-encoding chimeric gene, and obtaining progeny plants and seeds comprising said chimeric genes; or by transformation of a plant comprising a plant-expressible chimeric gene encoding a Cry2Ae protein insecticidal to Helicoverpa zea or Helicoverpa armigera with a second plant-expressible chimeric gene encoding a Cry1A, VIP3 or Cry1F protein insecticidal to Helicoverpa zea or Helicoverpa armigera, and obtaining progeny plants and seed comprising such at least two chimeric genes.

In another embodiment of this invention a method is provided for obtaining plants expressing at least two different insecticidal proteins, wherein said proteins do not share midgut binding sites in larvae of the species Helicoverpa zea or Helicoverpa armigera as can be determined in competition binding experiments using brush border membrane vesicles of said larvae, and wherein said proteins are: a) Cry2Ae protein insecticidal to Helicoverpa zea or Helicoverpa armigera and b) a Cry1A, VIP3 or Cry1F protein insecticidal to Helicoverpa zea or Helicoverpa armigera, particularly a VIP3 or Cry1A protein insecticidal to Helicoverpa zea or Helicoverpa armigera.

Also provided here is a method of sowing, planting, or growing plants protected against cotton bollworms, comprising chimeric genes expressing at least two different insecticidal proteins, wherein said proteins do not share binding sites in larvae of the species Helicoverpa zea or Helicoverpa armigera as determined in competition binding experiments using brush border membrane vesicles of said larvae, comprising the step of: sowing, planting, or growing plants comprising a chimeric gene encoding a Cry2Ae protein insecticidal to Helicoverpa zea or Helicoverpa armigera and a chimeric gene encoding a Cry1A, VIP3 or Cry1F protein insecticidal to Helicoverpa zea or Helicoverpa armigera, preferably a VIP3 or Cry1A protein insecticidal to Helicoverpa zea or Helicoverpa armigera.

Also provided herein is the use of at least two different insecticidal proteins in transgenic plants to prevent or delay insect resistance development in populations of Helicoverpa zea or Helicoverpa armigera, wherein said proteins do not share binding sites in the midgut of insects of said insect species, as can be determined by competition binding experiments, comprising expressing a Cry2Ae protein insecticidal to Helicoverpa zea or Helicoverpa armigera and a Cry1F, VIP3 or Cry1A protein insecticidal to Helicoverpa zea or Helicoverpa armigera in said transgenic plants, as well as the use of a chimeric gene encoding a Cry2Ae protein insecticidal to Helicoverpa zea or Helicoverpa armigera and a chimeric gene encoding a Cry1F, VIP3 or Cry1A protein insecticidal to Helicoverpa zea or Helicoverpa armigera, particularly a chimeric gene encoding a Cry2Ae protein insecticidal to Helicoverpa zea or Helicoverpa armigera and a chimeric gene encoding a VIP3 or Cry1A protein insecticidal to Helicoverpa zea or Helicoverpa armigera, for preventing or delaying insect resistance development in populations of the insect species Helicoverpa zea or Helicoverpa armigera to transgenic plants expressing insecticidal proteins to control said insect pest.

In one embodiment herein is provided the use of a Cry2Ae protein insecticidal to Helicoverpa zea or Helicoverpa armigera in combination with a Cry1A, VIP3 or Cry1F protein insecticidal to insects of said species, to prevent or delay resistance development of insects of said species to transgenic plants expressing heterologous insecticidal toxins, particularly when said use is by expression of said protein combination in plants.

Also provided herein is the use of plants comprising a Cry2Ae protein insecticidal to Helicoverpa zea or Helicoverpa armigera in a region where populations of said insect species have become resistant to plants comprising a Cry1F, VIP3 and/or Cry1A protein, wherein said use can comprise the sowing, planting or growing of plants comprising a Cry2Ae protein insecticidal to Helicoverpa zea or Helicoverpa armigera in said region, as well as the use of plants comprising a Cry1F, VIP3 and/or Cry1A protein insecticidal to Helicoverpa zea or Helicoverpa armigera in a region where populations of said insect species have become resistant to plants comprising a Cry2Ae protein, wherein said use can comprise the sowing, planting or growing of plants comprising a Cry1F, VIP3 and/or Cry1A protein insecticidal to Helicoverpa zea or Helicoverpa armigera in said region.

Also provided herein is the use of a chimeric gene encoding a Cry2Ae protein insecticidal to Helicoverpa zea or Helicoverpa armigera and a chimeric gene encoding a Cry1A, VIP3 or Cry1F protein insecticidal to Helicoverpa zea or Helicoverpa armigera, particularly a chimeric gene encoding a Cry2Ae protein insecticidal to Helicoverpa zea or Helicoverpa armigera and a chimeric gene encoding a Cry1A or VIP3 protein insecticidal to Helicoverpa zea or Helicoverpa armigera, in a method to obtain plants capable of expressing at least two different insecticidal proteins, wherein said proteins do not share binding sites in larvae of the species Helicoverpa zea or Helicoverpa armigera as can be determined in competition binding experiments, such as by using brush border membrane vesicles of said insect larvae.

In one embodiment of this invention, the use of a chimeric gene encoding a Cry2Ae protein insecticidal to Helicoverpa zea or Helicoverpa armigera is provided to obtain plants comprising at least two different insecticidal proteins, wherein said proteins do not share midgut binding sites in larvae of the species Helicoverpa zea or Helicoverpa armigera, as can be determined in competition binding experiments, such as by using brush border membrane vesicles of said insect larvae, wherein said Cry2Ae chimeric gene is present in plants also comprising a chimeric gene encoding a Cry1A, VIP3 or Cry1F protein insecticidal to Helicoverpa zea or Helicoverpa armigera.

In one embodiment, the above uses include the step of obtaining plants comprising such different insecticidal proteins by transformation of a plant with chimeric genes encoding said Cry2Ae and Cry1A, VIP3 or Cry1F proteins, and the obtaining of plants comprising such different insecticidal proteins by crossing plants comprising a chimeric gene encoding said Cry2Ae protein with plants comprising a chimeric gene encoding said Cry1A, VIP3 or Cry1F protein, and obtaining progeny plants and seeds of said plant comprising said chimeric genes.

The invention also provides for the use, the sowing, planting or growing of a refuge area with plants not comprising a Cry2, Cry1 or VIP3 protein insecticidal to Helicoverpa zea or Helicoverpa armigera, such as by sowing, planting or growing such plants in the same field or in the vicinity of the plants comprising the Cry2Ae, VIP3 and Cry1 protein described herein.

Also provided herein are the above uses or processes wherein the plants express the Cry2Ae, VIP3, Cry1F or Cry1A proteins at a high dose for Helicoverpa zea or Helicoverpa armigera.

Further provided herein is a process for growing, sowing or planting plants expressing a Cry protein or VIP3 protein for control of Helicoverpa armigera or Helicoverpa zea insects, comprising the step of planting, sowing or growing an insecticide sprayed structured refuge area of less than 20%, less than 15%, or less than 10% or an non-insecticide sprayed structured refuge area of less than 5%, of the planted field or in the vicinity of the planted field, or without planting, sowing or growing a structured refuge area in a field, wherein such structured refuge area is a location in the same field or is within 2 miles, within 1 mile or within 0.5 miles of a field, and which contains plants not comprising such Cry or VIP3 protein, wherein such plants expressing a Cry or VIP3 protein express a combination of a Cry2Ae protein insecticidal to said insect species, and a Cry1A, Cry1F or VIP3A protein, particularly a Cry2Ae and a Cry1Ab or Cry1Ac or VIP3A protein, preferably a Cry2Ae and Cry1Ab and VIP3 protein, insecticidal to said insect species. Also provided herein is a field of plants, particularly corn or cotton plants, comprising a structured refuge of less than 20%, of less than 15%, of less than 10%, or of less than 5%, or comprising no structured refuge, wherein said field is planted with plants expressing a combination of a Cry2Ae of Cry2Ab protein insecticidal to Helicoverpa armigera or Helicoverpa zea insects, and a Cry1A, Cry1F or VIP3A protein, particularly a Cry2Ae and a Cry1Ab, Cry1Ac or VIP3A protein, preferably a Cry2Ae and Cry1Ab and VIP3 protein, insecticidal to one of said insect species.

Also provided in one embodiment of this invention is the use of at least 2 insecticidal proteins binding specifically and saturably to binding sites in the midgut of Helicoverpa zea larvae, for delaying or preventing resistance development of such insect species to plants expressing insecticidal proteins, wherein one of said proteins in said plants is a Cry2A protein, such as a Cry2Ab protein, insecticidal to such insect species, and the other protein is a Cry1A, Cry1F or VIP3 protein insecticidal to such insect species, wherein such saturable binding is determined in a saturability assay using a fixed concentration of binding sites (i.e., BBMVs) to which increasing concentrations of labeled protein are added. Particularly, in such use the Cry1A protein is selected from the group of: a Cry1Ac, Cry1Ab, Cry1A.105, or a Cry1Ac or Cry1Ab hybrid protein, such as a protein encoded by any one of the cry1A coding regions referred to herein. Such Cry2Ab and Cry1A proteins do not compete for their (saturable and specific) binding sites in the midgut of such H. zea insect larvae, as can be measured in BBMV competition binding assays.

A Cry2Ae protein, as used herein, refers to an insecticidal Cry2Ae protein such as a full length Cry2Ae protein of SEQ ID No. 2 of WO 2002/057664 (Cry2Ae1, SEQ ID No. 1), a Cry2Ae toxic fragment or a protein comprising a Cry2Ae toxic fragment as described in of WO 2002/057664, such as a fusion protein of a Cry2Ae protein fragment with a chloroplast transit peptide or another peptide sequence insecticidal to H. zea or H. armigera, or is a protein insecticidal to H. zea or H. armigera comprising an amino acid sequence with at least 95, 97 or 99% sequence identity to the amino acid sequence of SEQ ID No. 1 herein or to SEQ ID No. 2 of WO 2002/057664, particularly in the part corresponding to the smallest toxic fragment, or is a protein encoded by the Cry2Ae coding region part of the Cry2Ae chimeric gene contained in cotton event EE-GH6 as described in the PCT patent application claiming priority to European patent application number 07075460 or 07075485 (unpublished), particularly any protein comprising the smallest toxic fragment of any one of such Cry2Ae proteins, or a variant of any one of such Cry2Ae proteins differing in 1-5 amino acids retaining toxicity to Helicoverpa zea or Helicoverpa armigera.

A Cry2Ab protein, as used herein, refers to any one of the Cry2Ab proteins of Crickmore et al. (1998), or www.lifesci.susx.ac.uk/home/Neil_Crickmore/Bt/insecticidal to H. zea or H. armigera, such as a full length Cry2Ab protein, a Cry2Ab toxic fragment, a Cry2Ab2 protein (SEQ ID No. 2 herein), or a protein comprising a Cry2Ab toxic fragment, such as a fusion protein of a Cry2Ab2 protein fragment with a chloroplast transit peptide or another peptide sequence retaining toxicity to Helicoverpa zea or Helicoverpa armigera, or is a protein insecticidal to Helicoverpa zea or Helicoverpa armigera comprising an amino acid sequence with at least 95, 97 or 99% sequence identity to SEQ ID No. 2 herein, or to the amino acid sequence of NCBI accession CAA39075 (Dankocsik et al., 1990), particularly in the part corresponding to the smallest toxic fragment, or is the protein encoded by the Cry2Ab2 coding region part of the Cry2Ab chimeric gene contained in cotton event 15985 as described in USDA-APHIS petition for non-regulated status 00-342-01p, the protein encoded by the Cry2Ab2 coding region part of the Cry2Ab chimeric gene contained in corn event MON89034 as described in USDA-APHIS petition for non-regulated status 06-298-01p, particularly any protein comprising the smallest toxic fragment of any one of such Cry2Ab proteins, or a variant of any one of such Cry2Ab proteins differing in 1-5 amino acids retaining toxicity to Helicoverpa zea or Helicoverpa armigera.

A Cry1F protein, as used herein, includes any protein comprising the smallest toxic fragment of the amino acid sequence of a Cry1F protein retaining toxicity to Helicoverpa zea or Helicoverpa armigera, such as the protein of NCBI accession AAA22347 (SEQ ID No. 10 of US 2005049410), or a Cry1Fa1 protein (SEQ ID No. 3). Also included in this definition are variants of the amino acid sequence in NCBI accession AAA22347 of SEQ ID No. 3, such as amino acid sequences having a sequence identity of at least 90% to SEQ ID No. 3 or to the Cry1F protein of NCBI accession AAA22347, as determined using pairwise alignments using the GAP program of the Wisconsin package of GCG (Madison, Wis., USA, version 10.2), particularly such identity is with the part corresponding to the smallest toxic fragment. A Cry1F protein, as used herein, includes the protein encoded by the Cry1F gene in Cry1F Cotton Event 281-24-236 (WO 2005/103266, see USDA APHIS petition for non-regulated status 03-036-01p), or in corn events TC1507 or TC-2675 (U.S. Pat. No. 7,288,643, WO 2004/099447, USDA APHIS petitions for non-regulated status 00-136-01p and 03-181-01p), particularly any protein comprising the smallest toxic fragment of any one of such Cry1F proteins, or a variant of any one of such Cry1F proteins differing in 1-5 amino acids with toxicity to Helicoverpa zea or Helicoverpa armigera.

In one embodiment in the invention, the VIP3 protein is a protein insecticidal to Helicoverpa zea or Helicoverpa armigera larvae, and which is any one of the VIP3 proteins listed in Crickmore et al. (2008), or any protein comprising the smallest toxic fragment of any one of these proteins. the VIP3 protein used is a VIP3A protein insecticidal to Helicoverpa zea or Helicoverpa armigera, such as the VIP3Aa1 protein of SEQ ID No. 4, the VIP3Af1 protein of SEQ ID No. 5, VIP3Aa19 (NCBI accession ABG20428, EPA experimental use permit factsheet 006499 (2007), SEQ ID No. 6 herein) or VIP3Aa20 protein (SEQ ID No. 7 herein) described herein, but also any protein comprising an insecticidal fragment or functional domain thereof, as well as any protein insecticidal to Helicoverpa zea or Helicoverpa armigera with a sequence identity of at least 70% with the VIP3Aa1 protein of SEQ ID No. 4, or NCBI accession AAC37036 (Estruch et al., 1996), particularly with its smallest toxic fragment, or with the VIP3Af1 protein of NCBI accession CAI43275 (SEQ ID No. 5 herein, SEQ ID No. 4 in WO03/080656), particularly with its smallest toxic fragment, as determined using pairwise alignments using the GAP program of the Wisconsin package of GCG, as well as a VIP3A protein insecticidal to Helicoverpa zea or Helicoverpa armigera selected from the group of: VIP3Ab, VIP3Ac, VIP3Ad, VIP3Ae, VIP3Af, VIP3Ag, or VIP3Ah, particularly the VIP3Af1, VIP3Ad1 or VIP3Ae1 proteins (NCBI accessions CAI43275 (ISP3a, SEQ ID No. 4 of WO 03/080656), CAI43276 (ISP3b, SEQ ID No. 6 in WO 03/080656), and CAI43277 (ISP3C, SEQ ID No. 2 of WO 03/080656), respectively) and insecticidal fragments, hybrids or variants thereof. In one embodiment, the VIP3 protein is the VIP3Aa19 protein (NCBI accession ABG20428, SEQ ID No. 6) introduced in cotton plants (e.g., in plants containing event COT102 described in WO 2004/039986, or in USDA APHIS petition for non-regulated status 03-155-01p) or the VIP3Aa20 protein (NCBI accession ABG20429, SEQ ID NO: 2 in WO 2007/142840, SEQ ID No. 7 herein) introduced in corn plants (e.g., event MIR162, USDA APHIS petition for non-regulated status 07-253-01p), or the VIP3A protein produced in cotton event COT202 or COT203 (WO 2005/054479 and WO 2005/054480, respectively), or a variant of any one of the above VIP3 proteins differing in 1-5 amino acids and retaining toxicity to Helicoverpa zea or Helicoverpa armigera.

A Cry1A protein, as used herein, refers to a Cry1Ac1 (SEQ ID No. 8), Cry1A.105 (SEQ ID No. 9) or a Cry1Ab1 (SEQ ID No. 10) protein, and includes any protein comprising the smallest toxic fragment of the amino acid sequence of a Cry1Ac, Cry1A.105 or Cry1Ab protein retaining toxicity to Helicoverpa zea or Helicoverpa armigera, such as any protein comprising the smallest toxic fragment of the protein in SEQ ID No. 8 or in NCBI accession AAA22331 (Cry1Ac; Adang et al., 1985), of the protein in SEQ ID No. 10 or in NCBI accession AAA22330 (Wabiko et al., 1986 (Cry1Ab)), or of the Cry1A.105 protein in SEQ ID No. 9 herein, encoded by the Cry1A transgene in corn event MON89034 (USDA APHIS petition for non-regulated status 06-298-01p, WO 2007/140256, SEQ ID NO: 2 or 4 in WO 2007/027777), or of the Cry1Ab protein encoded by the cry1Ab coding region in cotton event COT67B (USDA APHIS petition for non-deregulated status 07-108-01p, WO 2006/128573). Also included in this definition are variants of the amino acid sequence in NCBI accession AAA22331 (Cry1Ac1), NCBI accession AAA22330 (Cry1Ab, Wabiko et al., 1986), or the amino acid sequence of the Cry1A.105 protein described in USDA APHIS petition for non-regulated status 06-298-01p, such as proteins having an amino acid sequence identity of at least 90% with such a Cry1Ac, Cry1A.105 or Cry1Ab protein, particularly of SEQ ID Nos. 8, 9 or 10, more particularly in the part corresponding to the smallest toxic fragment, as determined using pairwise alignments using the GAP program of the Wisconsin package of GCG (Madison, Wis., USA, version 10.2), with the smallest toxic fragment of a Cry1A protein. Included herein as Cry1A proteins are the Cry1Ab protein encoded by SEQ ID NO:3 of U.S. Pat. No. 6,114,608, particularly the Cry1Ab protein encoded by the cry1Ab coding region in corn event MON810 (U.S. Pat. No. 6,713,259), USDA APHIS petition for non-deregulated status 96-017-01p and extensions thereof), the Cry1Ab protein encoded by the cry1Ab coding region in corn event Bt11 (USDA APHIS petition for non-deregulated status 95-195-01p, U.S. Pat. No. 6,114,608), the Cry1Ac protein encoded by the transgene in cotton event 3006-210-23 (U.S. Pat. No. 7,179,965, WO 2005/103266, USDA APHIS petition for non-deregulated status 03-036-02p), the Cry1Ab protein encoded by the cry1Ab coding region in cotton event COT67B (USDA APHIS petition for non-deregulated status 07-108-01p, WO 2006/128573), the Cry1Ab coding region contained in cotton event EE-GH5 described in PCT patent application PCT/EP2008/002667 (unpublished), the Cry1Ab coding region of SEQ ID No. 2 of U.S. Pat. No. 7,049,491, the Cry1A.105 protein encoded by the Cry1A transgene in corn event MON89034 (USDA APHIS petition for non-regulated status 06-298-01p, WO 2007/140256, SEQ ID NO: 2 or 4 in WO 2007/027777), the Cry1Ac-like protein encoded by the hybrid cry1Ac coding region in cotton event 15985 or cotton event 531, 757, or 1076 (USDA APHIS petition for non-regulated status 94-308-01p, the chimeric Cry1Ac protein encoded by the cry1A cotton event of WO 2002/100163), the cry1Ab protein encoded by the cry1Ab coding region in cotton events T342-142, 1143-14A, 1143-51B, CE44-69D, or CE46-02A of WO 2006/128568, WO 2006/128569, WO 2006/128570, WO 2006/128571, or WO 2006/128572 respectively (i.e., the protein encoded by the DNA of SEQ ID No. 7 in WO 2006/128568, WO 2006/128569, WO 2006/128571, or WO 2006/128572, or by the DNA of SEQ ID No. 5 in WO 2006/128570), or a protein comprising the smallest toxic fragment of any one of such Cry1A proteins, or a variant of any one of the above Cry1A proteins differing in 1-5 amino acids but retaining toxicity to H. zea or H. armigera.

Also provided herein are plants or seeds comprising at least 2 transgenes each encoding a different protein insecticidal to H. zea or armigera which proteins bind saturably and specifically to binding sites in the midgut of such insects, wherein said proteins do not compete for the same binding sites in such insects, and wherein said proteins are i) a Cry2A protein and ii) a Cry1A, Cry1F or VIP3 protein. In one embodiment said plants comprise transgenes encoding the proteins: i) Cry2Aa, Cry2Ab or Cry2Ae, and ii) Cry1Ab, Cry1Ac, Cry1Fa, or VIP3A, particularly a Cry2Ae protein and a Cry1Ab and/or VIP3A protein. In another embodiment said plants or seeds are corn or cotton plants or seeds containing a chimeric gene encoding a Cry1A, Cry1F or VIP3 protein and a chimeric gene encoding a Cry2A protein, particularly a Cry2Ae protein, wherein said plants or seeds contain a transformation event selected from the group consisting of: corn event MON89034, corn event MIR162, a corn event comprising a transgene encoding a Cry2Ae protein, corn event TC1507, corn event Bt11, corn event MON810, cotton event EE-GH6, cotton event COT102, cotton event COT202, cotton event COT203, cotton event T342-142, cotton event 1143-14A, cotton event 1143-51B, cotton event CE44-69D, cotton event CE46-02A, cotton event COT67B, cotton event 15985, cotton event 3006-210-23, cotton event 531, cotton event EE-GH5, cotton Event 281-24-236, all as defined further herein.

Also provided herein are plants comprising at least 3 transgenes each encoding a different protein insecticidal to H. zea or H. armigera which proteins bind saturably and specifically to binding sites in the midgut of such insects, wherein said proteins do not compete for the same binding sites in such insects, and wherein said plants contain a chimeric gene encoding a Cry1A or Cry1F protein, a chimeric gene encoding a Cry2A protein, and a chimeric gene encoding a VIP3A protein, and wherein the events are selected from the group as set forth in the above paragraphs.

In one embodiment of this invention, in the uses, methods or plant of the invention the Cry2Ae, Cry2Ab, VIP3, Cry1F or Cry1A chimeric genes are the chimeric genes contained in any one of the above corn or cotton events. In accordance with the invention is also included any one of the herein described uses, methods, plants or seeds wherein the term Cry2Ae is replaced by the term Cry2Aa or Cry2Ab, as well as any of the above uses, methods, plants or seeds involving a Cry2Ae, Cry2Aa or Cry2Ab protein wherein the binding of such Cry2A protein is specific and saturable to the midgut BBMVs of H. zea or H. armigera, particularly when saturable binding is determined in a direct saturability binding assay; preferably such uses, processes, plants or seeds wherein there is no biologically significant competition between the specific binding of any of said Cry2A protein and a Cry1A, Cry1F or VIP3 protein, in standard competition binding assays as described herein, in H. armigera or H. zea.

In the above plants, seeds, uses or methods of the current invention, preferred plants, such as for stacking or combining different chimeric genes in the same plants by crossing, are plants comprising any one of the above corn events or any one of the above cotton events, as well as their progeny or descendants comprising said Cry2A, and said VIP3 and/or Cry1 protein-encoding chimeric genes.

Plants or seeds as used herein include plants or seeds of any plant species significantly damaged by cotton bollworms, but particularly include corn, cotton, rice, soybean, sorghum, tomato, sunflower and sugarcane.

Further provided herein is a method for deregulating or for obtaining regulatory approval for planting or commercialization of plants expressing proteins insecticidal to H. zea or H. armigera, or for obtaining a reduction in structured refuge area containing plants not producing any protein insecticidal to H. zea or H. armigera, or for planting fields without a structured refuge area, such method comprising the step of referring to, submitting or relying on insect assay binding data showing that Cry2A proteins bind specifically and saturably to the insect midgut membrane of such insects, and that said Cry2A proteins do not compete with binding sites for Cry1A, Cry1F or VIP3 proteins in such insects, such as the data disclosed herein or similar data reported in another document. In one embodiment such Cry2A protein is a Cry2Aa, Cry2Ab or Cry2Ae protein and such Cry1A protein is a Cry1Ac, Cry1Ab, or Cry1A.105 protein, and said VIP3 protein is a VIP3Aa protein.

Further provided herein is a field planted with plants containing insecticidal proteins to protect said plants from Helicoverpa armigera or Helicoverpa zea insects, wherein said field has a structured refuge of less than 20%, or a structured refuge of less than 5%, or has no structured refuge in said field, and wherein said plants express a combination of a) a Cry2Ae protein insecticidal to said insect species and b) a Cry1A, Cry1F or VIP3A protein insecticidal to said insect species, in said plants. Said plants are preferably corn or cotton plants.

Also included herein are the above methods or plants, wherein besides the Cry or VIP3 proteins, also a Bt toxin enhancer protein is expressed in said plants, wherein said Bt toxin enhancer protein is a protein or a fragments thereof which is a part, preferably a part comprising or corresponding to the binding domain, of a Bt toxin receptor in an insect, such as a fragment of a cadherin-like protein. These Bt toxin enhancer proteins are fed to target insects together with one or more Bt insecticidal toxins such as Cry proteins. These Bt toxin enhancer proteins can enhance the toxin activity of the Bt insecticidal protein against the insect species that was the source of the receptor but also against other insect species. In one embodiment, said Bt toxin enhancer protein is a part of a midgut cell Bt toxin receptor of a H. zea or H. armigera insect.

### DETAILED DESCRIPTION OF THE INVENTION

Because of the success and the increasing number of plants comprising introduced insecticidal proteins such as Bt Cry or VIP3 proteins, resistance management is even more important now than in the past.

While the insecticidal spectrum of different insecticidal proteins derived from Bt or other bacteria, such as the Cry or VIP proteins, can be different, the major pathway of their toxic action is common. All Bt-derived insecticidal proteins used in transgenic plants, for which the mechanism of action has been studied in at least one target insect (e.g., Cry1 and VIP3 toxins), are proteolytically activated in the insect gut and interact with the midgut epithelium of sensitive species and cause lysis of the epithelial cells. In the pathway of toxic action of Cry proteins and VIP proteins, the specific binding of the toxin to receptor sites on the brush border membrane of these cells is a crucial feature (Hofmann et al., 1988; Lee et al., 2003). The binding sites are typically referred to as receptors, since the binding is saturable and with high affinity.

When two different insecticidal proteins share receptor binding sites in insects, they do not provide a good combination for insect resistance management purposes. Indeed, the most likely mechanism of resistance to insecticidal proteins such as Bt Cry proteins—and the only major mechanism found in field-developed insect resistance to Bt sprays so far—is receptor binding modification. Proteins that are highly similar in amino acid sequence often share receptor sites (e.g., the Cry1Ab and Cry1Ac proteins). But, even two different proteins having quite a different amino acid sequence may bind with high affinity to a common binding site in an insect species (such as, e.g., the Cry1Ab and Cry1F proteins in Plutella xylostella). Also, it has been found that two proteins that do not share binding sites in one insect species, may share a common binding site in another insect species (e.g., the Cry1Ac and Cry1Ba proteins were found to share a binding site in Chilo suppressalis by Fiuza et al. (1996) while they were found to bind to different binding sites in Plutella xylostella (Ballester et al. 1999)).

The current invention relates to Cry2A proteins that do not show competition for the Cry1F, VIP3 or Cry1A receptor in Helicoverpa zea or Helicoverpa armigera, making it most interesting to combine in the same plant at least a Cry2Ae, Cry2Aa or Cry2Ab protein with a VIP3, Cry1F or Cry1A protein, preferably at least a Cry2Ae protein and a Cry1Ab, Cry1Ac, Cry1A.105 or VIP3A protein, to prevent or delay the development of insect resistance to Helicoverpa zea or Helicoverpa armigera. This approach should ideally be part of a global approach for insect resistance management including, where desired or required, structured refuge areas and the expression of the proteins at a high dose for the target insect.

The binding sites which are referred to herein only refer to the specific binding sites for proteins insecticidal to H. zea or H. armigera, such as the Cry2Ae, Cry2Ab, VIP3A, Cry1Ac or Cry1Ab proteins. These are the binding sites to which a protein binds specifically, i.e., for which the binding of a labeled ligand (such as a Cry2Ae or VIP3A protein), to its binding site, can be displaced (or competed for) by an excess of non-labeled homologous ligand (a Cry2Ae or VIP3A protein, respectively). The terms binding site or receptor are used interchangeably herein and are equivalent. In one embodiment, the binding to such specific binding sites is saturable as measured in a direct saturability assay. As used herein, a “direct saturability assay” is an assay in which a fixed amount of receptor (in this case BBMV) is incubated with increasing amounts of labeled ligand. In case of saturable binding, a plateau—or at least a deviation from linearity—will be evident when the binding data are plotted (% binding on the Y-axis, concentration of labeled ligand on the X-axis), whereas in case of non-saturable binding, no plateau—or deviation from linearity—will be evident, but % binding keeps increasing linearly with increasing concentrations of labeled ligand. The plateau is the maximum binding that can be obtained in the experimental conditions because all the available specific binding sites have been occupied by the labeled ligand.

It is important when combining different insecticidal proteins in plants with the aim to delay or decrease insect resistance development of a target insect species, to check experimentally (i.e., by performing binding assays) in the target insect species if a proposed combination of different insecticidal proteins shares binding sites in the midgut of the target insect. In the current invention, when there is competition between two different insecticidal proteins for a single binding site (meaning when the binding data from competition binding experiments are plotted, both proteins reach the same plateau at the bottom of the competition curve), such proteins are not a useful combination in plants from an insect resistance management perspective. As used herein, when two different insecticidal proteins bind to two different binding sites, such proteins are useful from an insect resistance management perspective. As used herein, for proteins binding to different binding sites, competition of one protein for the binding site of another protein is not considered biologically significant (or, in other words, is considered biologically insignificant competition) if the competition takes place only at very high concentrations of the heterologous competitor (e.g., if 100 nM (or more) of the unlabeled heterologous competitor displaces only a minimal amount of bound labeled ligand (e.g., about 25% or less of the specific binding of the labeled ligand) as determined when the binding data are plotted (% binding vs. concentration of unlabeled ligand)). If a protein X binds only with low affinity (e.g., if 100 nM (or more) of the unlabeled heterologous competitor displaces only a minimal amount of bound labeled ligand (e.g., about 25% or less of the specific binding of the labeled ligand) as determined when the binding data are plotted (% binding vs. concentration of unlabeled ligand)) to the binding sites of a labeled protein Y, but there is no evidence of any different binding site in reciprocal binding assays using labeled protein X, both proteins effectively bind to the same binding site and hence are not suitable to be combined for resistance management purposes.

In this invention, measuring Cry or VIP3 protein binding by ligand blotting using denatured BBMV proteins is not deemed to be a reliable measure of the actual specific binding sites present in the midgut or in BBMV preparations (which can be measured in BBMV binding assays using radiolabeled, or biotinylated proteins, since binding is to non-denatured BBMV proteins in such assays), as (binding) characteristics of denatured proteins may be different from non-denatured proteins.

The methods and techniques for testing sharing of binding sites to insect larvae for a pair of different insecticidal proteins are well known in the art (see, e.g., Van Rie et al., 1989, Ferré et al., 1991). At first, one determines a pair of insecticidal proteins which are both insecticidal to the target insect, here H. zea or H. armigera. Brush border membrane vesicles (BBMV) are prepared from the midguts of Helicoverpa zea or Helicoverpa armigera using known procedures (see, e.g., Wolfersberger et al. 1987), and the specific binding of purified labeled protein (such as a Cry2Ae, Cry2Ab, VIP3 or Cry1 protein) to such BBMV is analyzed. Homologous competition assays are done to determine if the binding is specific (herein an excess of the same unlabeled protein is used as competitor for the labeled ligand), and heterologous competition assays are done to determine if another protein competes for the same binding site in these BBMV (herein an excess of a different, unlabeled protein is used as competitor for the labeled ligand).

In such heterologous competition assays, when no competition is found using labeled protein X and unlabeled protein Y as competitor, also the reciprocal experiment is done to confirm absence of competition, using the labeled protein Y and the unlabeled protein X as competitor. In homologous competition assays, the binding is specific if a significant part of the binding of labeled protein is competed for (or displaced by) the unlabeled protein (i.e., the homologous competitor)—the part of the binding which is not displaced or competed for by homologous ligand is considered non-specific binding. Labeling of the proteins, such as the Cry2Ae, Cry2Ab, VIP3 or Cry1 proteins used in this invention, can be done by the well known techniques of biotin-labeling, fluorescent labeling, or by radioactive labeling, such as by using Na¹²⁵Iodine (using known methods, e.g., Chloramine-T method).

In accordance with this invention, a “nucleic acid sequence” refers to a DNA or RNA molecule in single or double stranded form, preferably a DNA or RNA, particularly a DNA, encoding any of the proteins used in this invention. An “isolated nucleic acid sequence”, as used herein, refers to a nucleic acid sequence which is no longer in the natural environment where it was isolated from, e.g., the nucleic acid sequence in another bacterial host or in a plant nuclear genome.

As used herein “heterologous” proteins, such as when referring to the use of heterologous insecticidal proteins in plants, refers to proteins not present in such organism (such as a plant) in nature, particularly to proteins encoded by transgenes introduced into the genome of plants, wherein such proteins are derived from bacterial proteins.

In accordance with this invention, the terms “protein” or “polypeptide” are used interchangeably to refer to a molecule consisting of a chain of amino acids, without reference to any specific mode of action, size, three-dimensional structures or origin. Hence, a fragment or portion of a protein used in the invention is still referred to herein as a “protein”. An “isolated protein”, as used herein, refers to a protein which is no longer in its natural environment. The natural environment of the protein refers to the environment in which the protein could be found when the nucleotide sequence encoding it was expressed and translated in its natural environment, i.e., in the environment from which the nucleotide sequence was isolated. For example, an isolated protein can be present in vitro, or in another bacterial host or in a plant cell or it can be secreted from another bacterial host or from a plant cell.

As used herein, “insecticidal protein” should be understood as an intact protein or a part thereof which has insecticidal activity, particularly insecticidal to Helicoverpa zea or Helicoverpa armigera larvae. This can be a naturally-occurring protein or a chimeric or hybrid protein comprising parts of different insecticidal proteins (such as mixing domains from different proteins, or mixing parts of different proteins by using gene shuffling), or can be a variant having substantially the amino acid sequence of a bacterial protein but modified in some amino acids. In this regard, such an insecticidal protein can be a VIP or a Cry protein derived from Bt or other bacterial strains, or proteins encoded by mutant or recombinant genes as can be obtained by gene shuffling, mutagenesis, and the like from genes encoding Bt insecticidal proteins, such as Cry or VIP proteins.

As used herein, “protoxin” should be understood as the primary translation product of a full-length gene encoding an insecticidal protein, before any cleavage has occurred in the midgut. Typically, a VIP3 protoxin has a molecular weight of about 88 kD, a Cry1F or Cry1A protoxin has a molecular weight of about 130-140 kD, and a Cry2A protoxin has a molecular weight of about 60-70 kD.

As used herein, “toxin” or “smallest toxic fragment” should be understood as that part of an insecticidal protein, such as a Cry2A, VIP3 or Cry1F or Cry1A protein, which can be obtained by trypsin digestion or by proteolysis in (target insect, e.g., Helicoverpa zea or Helicoverpa armigera) midgut juice, and which still has insecticidal activity. Typically, a VIP3 or Cry1 toxin has a molecular weight of about 60-65 kD, and a Cry2A toxin has a molecular weight of about 50-58 kD on SDS-PAGE gel.

As used herein, a “VIP3 protein” or “VIP3”, refers to a protein insecticidal to Helicoverpa zea or Helicoverpa armigera larvae, and which is any one of the VIP3 proteins listed in Crickmore et al. (2008, see Table 3) on the VIP nomenclature website at: www.lifesci.susx.ac.uk/home/Neil_Crickmore/Bt/VIP.html, or any protein comprising the smallest toxic fragment of any one of these proteins. In one embodiment, this is a VIP3A protein insecticidal to Helicoverpa zea or Helicoverpa armigera, such as a VIP3Aa1 (NCBI accession AAC37036), VIP3Af1 (NCBI accession CAI43275), VIP3Aa19 (NCBI accession ABG20428) or VIP3Aa20 protein (NCBI accession ABG20429), but also any insecticidal fragments thereof, or proteins with a sequence identity of at least 70%, particularly at least 75%, 80%, 85%, 90%, 95%, 96%, 97%, 98% or 99% at the amino acid sequence level with the VIP3Aa1 protein of NCBI accession AAC37036, or the VIP3Af1 protein of NCBI accession CAI43275 (ISP3A, SEQ ID No. 4 in WO 03/080656), particularly with their smallest toxic fragment, as determined using pairwise alignments using the GAP program of the Wisconsin package of GCG (Madison, Wis., USA, version 10.2). The GAP program is used with the following parameters for the amino acid sequence comparisons: the ‘blosum62’ scoring matrix, a ‘gap creation penalty’ (or ‘gap weight’) of 8 and a ‘gap extension penalty’ (or ‘length weight’) of 2. In one embodiment, a VIP3 protein as used herein, is a VIP3A protein such as the VIP3Aa1 protein described in Estruch et al. (1996, NCBI accession AAC37036), as well as a VIP3A protein insecticidal to Helicoverpa zea or Helicoverpa armigera selected from the group of: VIP3Ab, VIP3Ac, VIP3Ad, VIP3Ae, VIP3Af, VIP3Ag, or VIP3Ah, particularly the VIP3Af1, VIP3Ad1 or VIP3Ae1 proteins (NCBI accessions CAI43275 (ISP3a, SEQ ID No. 4 of WO 03/080656), CAI43276 (ISP3b, SEQ ID No. 6 in WO 03/080656), and CAI43277 (ISP3C, SEQ ID No. 2 of WO 03/080656), respectively) and insecticidal fragments thereof. Of course, besides the naturally-occurring VIP3 protein, and proteins comprising an insecticidal fragment thereof, also hybrid or chimeric proteins made from VIP3 proteins retaining insecticidal activity to H. zea or H. armigera are included herein, such as the chimeric VIP3AcAa protein described in Fang et al. (2007), as well as VIP3 protein mutants or equivalents differing in some amino acids but retaining most or all of the H. zea or H. armigera toxicity of the parent molecule; such as VIP3 protein variants having some, preferably 5-10, particularly less than 5, amino acids added, replaced or deleted, preferably in the part corresponding to the smallest toxic fragment, without significantly changing the Helicoverpa zea or Helicoverpa armigera insecticidal activity of the protein, such as the VIP3Aa19 protein (NCBI accession ABG20428, EPA experimental use permit factsheet 006499 (2007)) introduced in cotton plants (e.g., in plants containing event COT102 described in WO 2004/039986, or in USDA APHIS petition for non-regulated status 03-155-01p) or the VIP3Aa20 protein (NCBI accession ABG20429, SEQ ID NO: 2 in WO 2007/142840) introduced in corn plants (e.g., event MIR162, USDA APHIS petition for non-regulated status 07-253-01p), or the VIP3A protein produced in cotton event COT202 or COT203 (WO 2005/054479 and WO 2005/054480, respectively), or a protein comprising the smallest toxic fragment of any one of such VIP3 proteins, or a variant of any one of such VIP3 proteins differing in 1-5 amino acids retaining toxicity to Helicoverpa zea or Helicoverpa armigera.

Also, in one embodiment of this invention, in the VIP3 protein of the current invention any putative native (bacterial) secretion signal peptide can be deleted or can be replaced by a Met amino acid or Met-Ala dipeptide, or by an appropriate signal peptide, such as a chloroplast transit peptide. Putative signal peptides can be detected using computer based analysis, using programs such as the program Signal Peptide search (SignaIP V1.1 or 2.0), using a matrix for prokaryotic gram-positive bacteria and a threshold score of less than 0.5, especially a threshold score of 0.25 or less (Von Heijne, Gunnar, 1986 and Nielsen et al., 1996).

A “Cry1F protein” or “Cry1F”, as used herein, includes any protein comprising the smallest toxic fragment of the amino acid sequence of a Cry1F protein retaining toxicity to Helicoverpa zea or Helicoverpa armigera, such as the protein of NCBI accession AAA22347 (SEQ ID No. 10 of US 2005049410), or a Cry1Fa protein. This includes hybrid or chimeric proteins comprising this smallest toxic fragment, or at least one of the structural domains, preferably at least 2 of the 3 structural domains, of a Cry1F protein, such as the hybrid proteins derived from Cry1F in WO 1999/024581. Also included in this definition are variants of the amino acid sequence in NCBI accession AAA22347, such as amino acid sequences having a sequence identity of at least 90%, 95%, 96%, 97%, 98% or 99% to the Cry1F protein of NCBI accession AAA22347 (SEQ ID No. 10 of US 2005049410), as determined using pairwise alignments using the GAP program of the Wisconsin package of GCG (Madison, Wis., USA, version 10.2), particularly such identity is with the part corresponding to the smallest toxic fragment. The GAP program is used with the following parameters for the amino acid sequence comparisons: the ‘blosum62’ scoring matrix, a ‘gap creation penalty’ (or ‘gap weight’) of 8 and a ‘gap extension penalty’ (or ‘length weight’) of 2. Preferably proteins having some, preferably 5-10, particularly less than 5, amino acids added, replaced or deleted without significantly decreasing the Helicoverpa zea or Helicoverpa armigera insecticidal activity of the protein, such as a Cry1F protein with one or more conservative amino acid substitutions for cloning purposes, are included in this definition. A Cry1F protein, as used herein, includes the protein encoded by the Cry1F genes in Cry1F Cotton Event 281-24-236 (WO 2005/103266, see USDA APHIS petition for non-regulated status 03-036-01p), or in corn events TC1507 or TC-2675 (U.S. Pat. No. 7,288,643, WO 2004/099447, USDA APHIS petitions for non-regulated status 00-136-01p and 03-181-01p), particularly a protein comprising the smallest toxic fragment of any one of such Cry1F proteins, or a variant of any one of such Cry1F proteins differing in 1-5 amino acids retaining toxicity to Helicoverpa zea or Helicoverpa armigera.

A “Cry2Ae” protein, as used herein, refers to an insecticidal Cry2Ae protein such as a full length Cry2Ae protein of SEQ ID No. 2 of WO 2002/057664, a Cry2Ae toxic fragment or a protein comprising a Cry2Ae toxic fragment as described in of WO 2002/057664, such as a fusion protein of a Cry2Ae protein fragment with a chloroplast transit peptide or another peptide sequence insecticidal to H. zea or H. armigera, or is a protein insecticidal to H. zea or H. armigera comprising an amino acid sequence with at least 95, 97 or 99% sequence identity to the amino acid sequence of SEQ ID No. 2 of WO 2002/057664, particularly in the part corresponding to the smallest toxic fragment, or is a protein encoded by the Cry2Ae gene contained in cotton event EE-GH6 as described in the PCT patent application claiming priority to European patent application number 07075460 or 07075485 (unpublished), or a protein comprising the smallest toxic fragment of any one of such Cry2Ae proteins, or a variant of any one of such Cry2Ae proteins differing in 1-5 amino acids retaining toxicity to Helicoverpa zea or Helicoverpa armigera.

A “Cry2Ab” protein, as used herein, refers to any one of the Cry2Ab proteins of Crickmore et al. (1998, 2008) or www.lifesci.susx.ac.uk/home/Neil_Crickmore/Bt/insecticidal to H. zea or H. armigera, such as a full length Cry2Ab protein, a Cry2Ab toxic fragment, or a protein comprising a Cry2Ab toxic fragment, such as a fusion protein of a Cry2Ab2 protein fragment with a chloroplast transit peptide or another peptide sequence retaining toxicity to Helicoverpa zea or Helicoverpa armigera, or is a protein insecticidal to Helicoverpa zea or Helicoverpa armigera comprising an amino acid sequence with at least 95, 97 or 99% sequence identity to the coding region of NCBI accession CAA39075 (Dankocsik et al., 1990), particularly in the part corresponding to the smallest toxic fragment, or is the protein encoded by the Cry2Ab2 gene contained in cotton event 15985 as described in USDA-APHIS petition for non-regulated status 00-342-01p, the protein encoded by the Cry2Ab2 gene contained in corn event MON89034 as described in USDA-APHIS petition for non-regulated status 06-298-01p, or a protein comprising the smallest toxic fragment of any one of such Cry2Ab proteins, or a variant of any one of such Cry2Ab proteins differing in 1-5 amino acids retaining toxicity to Helicoverpa zea or Helicoverpa armigera.

A “Cry1A” protein, as used herein, refers to a Cry1Ac, Cry1A.105 or a Cry1Ab protein, and includes any protein comprising the smallest toxic fragment of the amino acid sequence of a Cry1Ac, Cry1A.105 or Cry1Ab protein retaining toxicity to Helicoverpa zea or Helicoverpa armigera, such as the smallest toxic fragment of the protein in NCBI accession AAA22331 (Cry1Ac; Adang et al., 1985), NCBI accession AAA22330 (Wabiko et al., 1986 (Cry1Ab)), or the Cry1A.105 protein encoded by the Cry1A transgene in corn event MON89034 (USDA APHIS petition for non-regulated status 06-298-01p, WO 2007/140256, SEQ ID NO: 2 or 4 in WO 2007/027777), or the Cry1Ab protein encoded by the cry1Ab coding region in cotton event COT67B (USDA APHIS petition for non-deregulated status 07-108-01p, WO 2006/128573). This includes hybrid or chimeric proteins comprising this smallest toxic fragment or at least one of the structural domains, preferably at least 2 of the 3 structural domains, of a Cry1A protein such as Cry1Ab or Cry1Ac, e.g., the chimeric or hybrid Cry1A proteins with increased cotton bollworm activity, as described in U.S. Pat. No. 6,962,705 or U.S. Pat. No. 7,070,982. Also included in this definition are variants of the amino acid sequence in NCBI accession AAA22331 (Cry1Ac1), NCBI accession AAA22330 (Cry1Ab, Wabiko et al., 1986), or the amino acid sequence of the Cry1A.105 protein described in USDA APHIS petition for non-regulated status 06-298-01p, such as proteins having an amino acid sequence identity of at least 90%, 95%, 96%, 97%, 98% or 99% at the amino acid sequence level with such a Cry1Ac, Cry1A.105 or Cry1Ab protein, particularly in the part corresponding to the smallest toxic fragment, as determined using pairwise alignments using the GAP program of the Wisconsin package of GCG (Madison, Wis., USA, version 10.2), with the smallest toxic fragment of a Cry1A protein. The GAP program is used with the following parameters for the amino acid sequence comparisons: the ‘blosum62’ scoring matrix, a ‘gap creation penalty’ (or ‘gap weight’) of 8 and a ‘gap extension penalty’ (or ‘length weight’) of 2. Preferably proteins having some, preferably 5-10, particularly less than 5, amino acids added, replaced or deleted without significantly changing the Helicoverpa zea or Helicoverpa armigera insecticidal activity of the protein, such as a Cry1A protein with one or more conservative amino acid substitutions (e.g., for gene cloning purposes), are included in this definition.

Examples of Cry1A proteins for use in this invention include the Cry1Ab protein encoded by SEQ ID NO:3 of U.S. Pat. No. 6,114,608, particularly the Cry1Ab protein encoded by the cry1Ab coding region in corn event MON810 (U.S. Pat. No. 6,713,259), USDA APHIS petition for non-deregulated status 96-017-01p and extensions thereof), the Cry1Ab protein encoded by the cry1Ab coding region in corn event Bt11 (USDA APHIS petition for non-deregulated status 95-195-01p, U.S. Pat. No. 6,114,608), the Cry1Ac protein encoded by the transgene in cotton event 3006-210-23 (U.S. Pat. No. 7,179,965, WO 2005/103266, USDA APHIS petition for non-deregulated status 03-036-02p), the Cry1Ab protein encoded by the cry1Ab coding region in cotton event COT67B (USDA APHIS petition for non-deregulated status 07-108-01p, WO 2006/128573), the Cry1Ab coding region contained in cotton event EE-GH5 described in PCT patent application PCT/EP2008/002667 (unpublished), the Cry1Ab coding region of SEQ ID No. 2 of U.S. Pat. No. 7,049,491, the Cry1A.105 protein encoded by the Cry1A transgene in corn event MON89034 (USDA APHIS petition for non-regulated status 06-298-01p, WO 2007/140256, SEQ ID NO: 2 or 4 in WO 2007/027777), the Cry1Ac-like protein encoded by the hybrid cry1Ac coding region in cotton event 15985 or cotton event 531, 757, or 1076 (USDA APHIS petition for non-regulated status 94-308-01p, the chimeric Cry1Ac protein encoded by the cry1A cotton event of WO 2002/100163), the cry1Ab protein encoded by the cry1Ab coding region in cotton events T342-142, 1143-14A, 1143-51B, CE44-69D, or CE46-02A of WO 2006/128568, WO 2006/128569, WO 2006/128570, WO 2006/128571, or WO 2006/128572 respectively (i.e., the protein encoded by the DNA of SEQ ID No. 7 in WO 2006/128568, WO 2006/128569, WO 2006/128571, or WO 2006/128572, or by the DNA of SEQ ID No. 5 in WO 2006/128570). In one embodiment of this invention, a Cry1Ab or a Cry1A.105 protein, or a protein comprising the smallest toxic fragment thereof, from this above list is used, or a protein comprising the smallest toxic fragment of any one of such Cry1A proteins, or a variant of any one of such Cry1A proteins differing in 1-5 amino acids retaining toxicity to Helicoverpa zea or Helicoverpa armigera.

In the current invention, it has been found that Cry2Ae and Cry2Aa proteins compete for the same binding site as the Cry2Ab protein in H. armigera, and that this binding site is different from (i.e. not shared with) the binding site of Cry1Ac in Helicoverpa zea and Helicoverpa armigera. Also, Cry1Ac did not compete for the binding of Cry2Ab in these insect species. Also, it has already been reported that Cry1F and Cry1Ac, and Cry1Ac and Cry1Ab share binding sites in Helicoverpa zea or Helicoverpa armigera (e.g., Hernandez and Ferre, 2005, Karim et al., 2000b; Estela et al., 2004). Hence, Cry1Ab, Cry1F and Cry1Ac bind to a binding site that is different from the binding site of Cry2Ae or Cry2Aa in H. zea or H. armigera. Also, it has been reported that the VIP3A protein binds to a different binding site than Cry2Ab (Lee et al., 2006). Since Cry2Aa and Cry2Ae share a common binding site with Cry2Ab, Cry2Ae or Cry2Aa proteins bind to a different binding site than the VIP3A protein in H. zea and H. armigera. Although Cry1F proteins generally have a lower activity to these insect species compared to the Cry1A, VIP3A or Cry2A proteins tested, they are amongst the most widely used Cry1 proteins in plants, and since they do not share binding sites with Cry2A proteins, they can also be used for insect resistance management, certainly if the plants can provide for sufficiently high levels of expression of the Cry1F protein. Some Cry1F-derived proteins have a higher intrinsic activity to H. zea or H. armigera, and these are a more preferred Cry1F protein in this invention. When there is a choice between a Cry1F and a Cry1A protein to combine (by crossing plants expressing a single insecticidal protein or by transformation) with a Cry2A protein in a given plant species, a Cry1A protein, such as a Cry1Ab or Cry1A.105 protein, will be the better choice to delay or prevent resistance development to Helicoverpa zea or Helicoverpa armigera, given their higher intrinsic toxicity to these insect species.

Bt Cry proteins such as Cry1F, Cry2A and Cry1A proteins are expressed as protoxins in their native host cells (Bacillus thuringiensis), which are converted into the toxin form by proteolysis in the insect gut. A Cry1F, Cry2A or Cry1A protein, as used herein, refers to either the full protoxin or the toxin, or any intermediate form with insecticidal activity. In one embodiment, a Cry1F protein includes a protein comprising the amino acid sequence of NCBI accession AAA22347 from amino acid position 29 to amino acid position 604, and a Cry1A protein includes a protein comprising the amino acid sequence of NCBI accession AAA22331 (Cry1Ac1; Adang et al., 1985) from amino acid position 29 to 607, comprising the amino acid sequence of NCBI accession AAA22330 (Cry1Ab, Wabiko et al., 1986) from amino acid position 29 to amino acid position 607, or comprising the amino acid sequence of Fig. IV-1 in USDA APHIS petition for non-regulated status 06-298-01p from amino acid position 29 to amino acid position 607. As used herein, a Cry2A protein includes a protein comprising the amino acid sequence of SEQ ID No. 2 of U.S. Pat. No. 7,265,269 from amino acid position 50 to 625, a protein comprising the amino acid sequence of Fig. IV-2 in USDA APHIS petition for non-regulated status 06-298-01p from amino acid position 81 to 746, or a protein comprising the amino acid sequence of NCBI accession CAA39075 from amino acid position 50 to amino acid position 626.

A “Cry1” protein, as used herein, refers to a Cry1F or Cry1A protein as defined above. A “Cry2A” protein, as used herein, refers to a Cry2Ae or Cry2Ab protein as defined herein, but can also refer to any Cry2A protein in Crickmore et al. (2008), such as a Cry2Aa protein, insecticidal to H. zea or H. armigera. A VIP3 or cry1 “gene” or “DNA”, as used herein, refers to a DNA encoding a VIP3 or Cry1 protein in accordance with this invention. A gene can be naturally occurring, artificial (modified) or synthetic in whole or in part.

The term “event”, as used herein, refers to a specific integration of one or more transgenes at a specific location in the plant genome, which can be considered as a part of DNA containing the inserted sequences and the flanking plant sequences. Such an event can be crossed into many other plants of the same species or in plants of a different species allowing intercrossing with the plants containing the event by breeding techniques, including techniques such as embryo rescue.

As used herein “comprising” is to be interpreted as specifying the presence of the stated features, integers, steps or components as referred to, but does not preclude the presence or addition of one or more features, integers, steps or components, or groups thereof. Thus, the term “DNA/protein comprising the sequence or region X”, as used herein, refers to a DNA or protein including or containing at least the sequence or region X, so that other nucleotide or amino acid sequences can be included at the 5′ (or N-terminal) and/or 3′ (or C-terminal) end, e.g. (the nucleotide sequence of) a transit peptide, and/or a 5′ or 3′ leader sequence.

A VIP3 or Cry protein-encoding “chimeric gene”, as used herein, refers to a VIP3 or Cry-encoding DNA (or coding region) having 5′ and/or 3′ regulatory sequences, at least a 5′ regulatory sequence or promoter, different from the naturally-occurring bacterial 5′ and/or 3′ regulatory sequences which drive the expression of the VIP3 or Cry protein in its native host cell, e.g., a VIP3 or cry DNA operably-linked to a plant-expressible promoter such that said chimeric gene can be expressed in the plants containing it. The chimeric gene need not be expressed the entire time or in every cell of the plant, e.g., expression can be induced by insect feeding or wounding using a wound-induced promoter, or expression can be localized in those plant parts mostly attacked by insects such as Helicoverpa zea or Helicoverpa armigera larvae, particularly those most valuable for the grower or farmer, e.g., the leaves and ears of a corn plant, or the leaves and bolls of cotton plants, or the leaves and pods of soybean plants. Hence, a plant expressing a VIP3, Cry2A, Cry1F or Cry1A protein as used herein refers to a plant containing the necessary plant-expressible chimeric gene encoding such a protein, so that the protein is expressed in the relevant tissues or at the relevant time periods, which need not be in all plant tissues or need not be all the time.

For the purpose of this invention the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other is regarded as a position with non-identical residues. To calculate sequence identity between two sequences for the purpose of this invention, the GAP program, which uses the Needleman and Wunsch algorithm (1970) and which is provided by the Wisconsin Package, Version 10.2, Genetics Computer Group (GCG), 575 Science Drive, Madison, Wis. 53711, USA, is used. The GAP parameters used are a gap creation penalty=50 (nucleotides)/8 (amino acids), a gap extension penalty=3 (nucleotides)/2 (amino acids), and a scoring matrix “nwsgapdna” (nucleotides) or “blosum62” (amino acids).

GAP uses the Needleman and Wunsch global alignment algorithm to align two sequences over their entire length, maximizing the number of matches and minimizes the number of gaps. The default parameters are a gap creation penalty=50 (nucleotides)/8 (proteins) and gap extension penalty=3 (nucleotides)/2 (proteins). For nucleotides the default scoring matrix used is “nwsgapdna” and for proteins the default scoring matrix is “blosum62” (Henikoff & Henikoff, 1992).

DNAs included herein as a VIP3 or Cry DNA are those DNAs that encode a VIP3 or Cry protein, or a variant or hybrid thereof, insecticidal to H. zea or H. armigera, and that hybridizes under stringent hybridization conditions to a DNA that can encode a VIP3 or Cry protein. “Stringent hybridization conditions”, as used herein, refers particularly to the following conditions: immobilizing the relevant DNA on a filter, and prehybridizing the filters for either 1 to 2 hours in 50% formamide, 5% SSPE, 2×Denhardt's reagent and 0.1% SDS at 42° C. or 1 to 2 hours in 6×SSC, 2×Denhardt′ s reagent and 0.1% SDS at 68° C. The denatured (Digoxigenin- or radio-) labeled probe is then added directly to the prehybridization fluid and incubation is carried out for 16 to 24 hours at the appropriate temperature mentioned above. After incubation, the filters are then washed for 30 minutes at room temperature in 2×SSC, 0.1% SDS, followed by 2 washes of 30 minutes each at 68° C. in 0.5×SSC and 0.1% SDS. An autoradiograph is established by exposing the filters for 24 to 48 hours to X-ray film (Kodak XAR-2 or equivalent) at −70° C. with an intensifying screen. [20×SSC=3M NaCl and 0.3M sodiumcitrate; 100×Denhart's reagent=2% (w/v) bovine serum albumin, 2% (w/v) FicollTM and 2% (w/v) polyvinylpyrrolidone; SDS=sodium dodecyl sulfate; 20×SSPE=3.6M NaCl, 02M Sodium phosphate and 0.02M EDTA pH7.7]. Of course, equivalent conditions and parameters can be used in this process while still retaining the desired stringent hybridization conditions.

“Insecticidal activity” of a protein, as used herein, means the capacity of a protein to kill insects when such protein is fed to insects, preferably by expression in a recombinant host such as a plant. It is understood that a protein has insecticidal activity if it has the capacity to kill the insect during at least one of its developmental stages, preferably the larval stage.

A population of insect species that “has developed resistance” or “has become resistant” to plants expressing an insecticidal protein (which plants formerly controlled or killed populations of said insect), as used herein, refers to the detection of repeated, significant unacceptable yield damage in such plants, caused by such insect population as compared to the level of yield damage of such plants by the same insect species when such plants were first introduced. This has to be confirmed to check that the plants are indeed producing the insecticidal protein (i.e., they are not non-transgenic plants), and that members of this insect population indeed need a higher amount of insecticidal protein to be controlled or killed. In other words, such plants to which an insect population has become resistant no longer produce an insect-controlling amount (as defined herein) or are no longer insecticidal for such insect species population. As such, “insect resistance development” as used herein, leads to an increased plant damage that is detected. In one embodiment, insect resistance of an insect species population is readily observed if insects from such population can complete their life cycle on such plants, and continue to damage the plants instead of being arrested in their growth and feeding habits because of the insecticidal proteins produced in such plants—in an extreme form of insect resistance such plant can be as damaged as conventional non-transgenic plants with the same genetic background by an insect attack. In one embodiment, the binding to Cry or VIP3 proteins to such resistant insects can be analyzed in (standard) competition binding assays using BBMV of H. zea or H. armigera, to confirm that resistance is due to binding site modification.

Also included herein are the above methods, uses, plants or plant cells, wherein besides the Cry or VIP3 proteins, also a Bt toxin enhancer protein is expressed in said plants, wherein said Bt toxin enhancer protein is a protein or a fragment thereof which is a part, preferably a part comprising or corresponding to the binding domain, of a Bt toxin receptor in an insect, such as a fragment of a cadherin-like protein. Such proteins are shown in published US patent application 20090018075. These Bt toxin enhancer proteins are fed to target insects together with one or more Bt insecticidal toxins such as Cry proteins. These Bt toxin enhancer proteins can enhance the toxic activity of the Bt insecticidal protein against the insect species that was the source of the receptor but also against other insect species. In one embodiment, said Bt toxin enhancer protein is a part of a midgut cell Bt toxin receptor of a H. zea or H. armigera insect.

“H. zea” as used herein, refers to Helicoverpa zea (Boddie), an important Lepidopteran pest insect, also known as the (American) cotton bollworm, the corn earworm or the tomato fruitworm, sorghum headworm, or vetchworm. This insect is an important pest in corn, cotton and tomato, but also attacks plants like artichoke, asparagus, cabbage, cantaloupe, collard, cowpea, cucumber, eggplant, lettuce, lima bean, melon, okra, pea, pepper, potato, pumpkin, snap bean, spinach, squash, sweet potato, watermelon, alfalfa, clover, flax, oat, millet, rice, sorghum, soybean, sugarcane, sunflower, tobacco, vetch, and wheat.

“H. armigera”, as used herein, refers to Helicoverpa armigera (Hübner), an important Lepidopteran pest insect, which is also known as the (African) cotton bollworm, tomato grubworm, tobacco budworm, corn earworm, old world bollworm, or scarce bordered straw, and is one of the most polyphagous insect pests with strong mobility. This insect is an important pest in corn and cotton, but it also attacks plants like tobacco, sunflower, linseed, soybean, Lucerne, peas such as pigeonpea or chickpea, chili, okra, besides carnations, geraniums and other ornamental or flower crops, fruits, and vegetables such as cabbage, aubergines, peppers, tomato, and cucumber.

“Cotton bollworm(s)” or “bollworm(s)”, as used herein in a general sense, refers to H. zea and/or H. armigera.

“Insect-controlling amounts” of a protein, as used herein, refers to an amount of protein which is sufficient to limit damage on a plant, caused by insects (e.g. insect larvae) feeding on such plant, to commercially acceptable levels, e.g. by killing the insects or by inhibiting the insect development, fertility or growth in such a manner that they provide less damage to a plant and plant yield is not significantly adversely affected.

In one embodiment of this invention, the VIP3 and/or Cry protein of the invention, are expressed at a high dose in the plants used in the invention. ‘High dose’ expression, as used herein when referring to the plants used in the invention, refers to a concentration of the insecticidal protein in a plant (measured by ELISA as a percentage of the total soluble protein, which total soluble protein is measured after extraction of soluble proteins in a standard extraction buffer using Bradford analysis (Bio-Rad, Richmond, Calif.; Bradford, 1976)) which kills at least 95% of insects in a developmental stage of the target insect which is significantly less susceptible, preferably at least 25 times less susceptible to the insecticidal protein than the first larval stage of the insect (as can be analyzed in standard insecticidal protein bio-assays), and can thus can be expected to ensure full control of the target insect species.

A “structured refuge” as used herein, refers to a part of the field or land of a grower or farmer that is otherwise planted with Bt-plants, but which is planted with plants not containing a Bt transgene (as compared to using weeds or other non-Bt plants around a farmer's fields as a natural refuge).

General procedures for the evaluation and exploitation of at least two insecticidal genes for prevention of the development, in a target insect, of resistance to transgenic plants expressing those genes can be found in published European patent application EP408403. Definitions used in the field of receptor binding analysis can be found at: www.unmc.edu/Pharmacology/receptortutorial/definitions/definitions.htm

In accordance with this invention, the binding of Cry proteins to the brush border membrane of the midgut cells of Helicoverpa zea or Helicoverpa armigera insect larvae has been investigated. The brush border membrane is the primary target of the VIP3 or Cry proteins, and membrane vesicles, preferentially derived from the insect midgut brush border membrane (named BBMV herein, for brush border membrane vesicles), can be obtained according to procedures known in the art, e.g., Wolfersberger et al. (1987).

This invention involves the combined expression of at least two insecticidal protein genes in transgenic plants to delay or prevent resistance development in populations of the target insect Helicoverpa zea or Helicoverpa armigera. The genes are inserted in a plant cell genome, preferably in its nuclear genome, so that the inserted genes are downstream of, and operably linked to, a promoter which can direct the expression of the genes in plant cells.

In one embodiment of this invention is provided a plant with a lasting resistance to Helicoverpa zea or Helicoverpa armigera, said plant comprising a chimeric gene encoding a Cry2A protein, such as a Cry2Ab or Cry2Ae protein, insecticidal to Helicoverpa zea or Helicoverpa armigera, and a chimeric gene encoding a Cry1A, VIP3 and/or Cry1F protein, preferably a Cry1Ab, VIP3A or a Cry1A.105 protein as defined above, insecticidal to Helicoverpa zea or Helicoverpa armigera.

Provided herein is also a method of controlling Helicoverpa zea or Helicoverpa armigera infestation in transgenic plants while securing a slower buildup of Helicoverpa zea or Helicoverpa armigera insect resistance development to said plants, comprising expressing a combination of a) a Cry2Ae protein insecticidal to said insect species and b) a Cry1A, Cry1F or VIP3A protein insecticidal to said insect species, in said plants, as well as a method for preventing or delaying insect resistance development in populations of the insect species Helicoverpa zea or Helicoverpa armigera to transgenic plants expressing insecticidal proteins to control said insect pest, comprising expressing a Cry2Ae protein insecticidal to Helicoverpa zea or Helicoverpa armigera in combination with a Cry1A, Cry1F of VIP3A protein insecticidal to Helicoverpa zea or Helicoverpa armigera in said plants.

In one embodiment of this invention, a method is provided to control Helicoverpa zea or Helicoverpa armigera in a region where populations of said insect species have become resistant to plants expressing a VIP3A, Cry1A or a Cry1F protein, comprising the step of sowing or planting in said region, plants expressing at least a Cry2Ae protein insecticidal to Helicoverpa zea or Helicoverpa armigera. Further provided herein is a method to control Helicoverpa zea or Helicoverpa armigera in a region where populations of said insect have become resistant to plants expressing a Cry2Ae protein, comprising the step of sowing or planting in said region, plants expressing a Cry1F, VIP3, or Cry1A protein insecticidal to Helicoverpa zea or Helicoverpa armigera.

Also provided in accordance with this invention is a method for obtaining plants comprising chimeric genes encoding at least two different insecticidal proteins, wherein said proteins do not share binding sites in larvae of the species Helicoverpa zea or Helicoverpa armigera as determined in competition binding experiments using brush border membrane vesicles of said insect larvae, comprising the step of obtaining plants comprising a plant-expressible chimeric gene encoding a Cry2Ae protein insecticidal to Helicoverpa zea or Helicoverpa armigera and a plant-expressible chimeric gene encoding a Cry1A, VIP3 or Cry1F protein insecticidal to Helicoverpa zea or Helicoverpa armigera.

Also provided here is a method of sowing, planting, or growing plants protected against cotton bollworms, comprising chimeric genes expressing at least two different insecticidal proteins, wherein said proteins do not share binding sites in larvae of the species Helicoverpa zea or Helicoverpa armigera as determined in competition binding experiments using brush border membrane vesicles of said larvae, comprising the step of: sowing, planting, or growing plants comprising a chimeric gene encoding a Cry2Ae protein insecticidal to Helicoverpa zea or Helicoverpa armigera and a chimeric gene encoding a Cry1A, VIP3 or Cry1F protein insecticidal to Helicoverpa zea or Helicoverpa armigera, preferably a VIP3 or Cry1A protein insecticidal to Helicoverpa zea or Helicoverpa armigera.

Also provided herein is the use of at least two different insecticidal proteins in transgenic plants to prevent or delay insect resistance development in populations of Helicoverpa zea or Helicoverpa armigera, wherein said proteins do not share binding sites in the midgut of insects of said insect species, as can be determined by competition binding experiments, comprising expressing a Cry2Ae protein insecticidal to Helicoverpa zea or Helicoverpa armigera and a Cry1F, VIP3 or Cry1A protein insecticidal to Helicoverpa zea or Helicoverpa armigera in said transgenic plants, as well as the use of a chimeric gene encoding a Cry2Ae protein insecticidal to Helicoverpa zea or Helicoverpa armigera and a chimeric gene encoding a Cry1F, VIP3 or Cry1A protein insecticidal to Helicoverpa zea or Helicoverpa armigera, particularly a chimeric gene encoding a Cry2Ae protein insecticidal to Helicoverpa zea or Helicoverpa armigera and a chimeric gene encoding a VIP3 or Cry1A protein insecticidal to Helicoverpa zea or Helicoverpa armigera, for preventing or delaying insect resistance development in populations of the insect species Helicoverpa zea or Helicoverpa armigera to transgenic plants expressing insecticidal proteins to control said insect pest.

In one embodiment herein is provided the use of a Cry2Ae protein insecticidal to Helicoverpa zea or Helicoverpa armigera in combination with a Cry1A, VIP3 or Cry1F protein insecticidal to insects of said species, to prevent or delay resistance development of insects of said species to transgenic plants expressing heterologous insecticidal toxins, particularly when said use is by expression of said proteins in plants.

Also provided herein is the use of plants comprising a Cry2Ae protein insecticidal to Helicoverpa zea or Helicoverpa armigera in a region where populations of said insect species have become resistant to plants comprising a Cry1F, VIP3 and/or Cry1A protein, wherein said use can comprise the sowing, planting or growing of plants comprising a Cry2Ae protein insecticidal to Helicoverpa zea or Helicoverpa armigera in said region, as well as the use of plants comprising a Cry1F, VIP3 and/or Cry1A protein insecticidal to Helicoverpa zea or Helicoverpa armigera in a region where populations of said insect species have become resistant to plants comprising a Cry2Ae protein, wherein said use can comprise the sowing, planting or growing of plants comprising a Cry1F, VIP3 and/or Cry1A protein insecticidal to Helicoverpa zea or Helicoverpa armigera in said region.

The invention also provides for the use, the sowing, planting or growing of a refuge area with plants not comprising a Cry2, Cry1 or VIP3 protein insecticidal to Helicoverpa zea or Helicoverpa armigera, such as by sowing, planting or growing such plants in the same field or in the vicinity of the plants comprising the Cry2Ae, VIP3 and Cry1 protein described herein.

Also provided herein are the above uses or processes wherein the plants express the Cry2Ae, VIP3, Cry1F or Cry1A proteins at a high dose for Helicoverpa zea or Helicoverpa armigera, as such high dose is defined herein.

Further provided herein is a process for growing, sowing or planting plants expressing a Cry protein or VIP3 protein for control of Helicoverpa armigera or Helicoverpa zea insects, comprising the step of planting, sowing or growing an insecticide sprayed structured refuge area of less than 20%, or an non-insecticide sprayed structured refuge area of less than 5%, of the planted field or in the vicinity of the planted field, or without planting, sowing or growing a structured refuge area in a field, wherein such structured refuge area is a location in the same field or is within 2 miles, within 1 mile or within 0.5 miles of a field, and which contains plants not comprising such Cry or VIP3 protein, wherein such plants expressing a Cry or VIP3 protein express a combination of a Cry2Ae protein insecticidal to said insect species, and a Cry1A, Cry1F or VIP3A protein, particularly a Cry2Ae and a Cry1Ab or Cry1Ac or VIP3A protein, preferably a Cry2Ae and Cry1Ab and VIP3 protein, insecticidal to said insect species.

Also provided in one embodiment of this invention is the use of at least 2 insecticidal proteins binding specifically and saturably to binding sites in the midgut of Helicoverpa zea larvae, for delaying or preventing resistance development of such insect species to plants expressing insecticidal proteins, wherein one of said proteins in said plants is a Cry2A protein, such as a Cry2Ab protein, insecticidal to such insect species, and the other protein is a Cry1A, Cry1F or VIP3 protein insecticidal to such insect species, wherein such saturable binding is determined in a saturability assay using a fixed concentration of binding sites (i.e., BBMVs) to which increasing concentrations of labeled protein are added. Particularly, in such use the Cry1A protein is selected from the group of: a Cry1Ac, Cry1Ab, Cry1A.105, or a Cry1Ac or Cry1Ab hybrid protein, such as a protein encoded by any one of the cry1A coding regions referred to herein. Such Cry2Ab and Cry1A proteins do not compete for their (saturable and specific) binding sites in the midgut of such H. zea insect larvae, as can be measured in BBMV competition binding assays.

Also provided herein are plants or seeds comprising at least 2 transgenes each encoding a different protein insecticidal to H. zea or armigera which proteins bind saturably and specifically to binding sites in the midgut of such insects, wherein said proteins do not compete for the same binding sites in such insects, and wherein said proteins are i) a Cry2A protein and ii) a Cry1A, Cry1F or VIP3 protein. In one embodiment said plants comprise transgenes encoding the proteins: i) Cry2Aa, Cry2Ab or Cry2Ae, and ii) Cry1Ab, Cry1Ac, Cry1Fa, or VIP3A, particularly a Cry2Ae protein and a Cry1Ab and/or VIP3A protein. In another embodiment said plants or seeds are corn or cotton plants or seeds containing a chimeric gene encoding a Cry1A, Cry1F or VIP3 protein and a chimeric gene encoding a Cry2A protein, particularly a Cry2Ae protein, wherein said plants or seeds contain a transformation event selected from the group consisting of: corn event MON89034, corn event MIR162, a corn event comprising a transgene encoding a Cry2Ae protein, corn event TC1507, corn event Bt11, corn event MON810, cotton event EE-GH6, cotton event COT102, cotton event COT202, cotton event COT203, cotton event T342-142, cotton event 1143-14A, cotton event 1143-51B, cotton event CE44-69D, cotton event CE46-02A, cotton event COT67B, cotton event 15985, cotton event 3006-210-23, cotton event 531, cotton event EE-GH5, cotton Event 281-24-236, all as defined further herein.

Also provided herein are plants comprising at least 3 transgenes each encoding a different protein insecticidal to H. zea or H. armigera which proteins bind saturably and specifically to binding sites in the midgut of such insects, wherein said proteins do not compete for the same binding sites in such insects, and wherein said plants contain a chimeric gene encoding a Cry1A or Cry1F protein, a chimeric gene encoding a Cry2A protein, and a chimeric gene encoding a VIP3A protein, and wherein the events are selected from the group as set forth in the above paragraphs.

In one embodiment of this invention, in the uses, methods or plant of the invention the Cry2Ae, Cry2Ab, VIP3, Cry1F or Cry1A chimeric genes are the chimeric genes contained in any one of the above specific corn or cotton events. In accordance with the invention is also included any one of the herein described uses, methods, plants or seeds wherein the term Cry2Ae is replaced by the term Cry2Aa or Cry2Ab, as well as any of the above uses, methods, plants or seeds involving a Cry2Ae, Cry2Aa or Cry2Ab protein wherein the binding of such Cry2A protein is specific and saturable to the midgut BBMVs of H. zea or H. armigera, particularly when saturable binding is determined in a direct saturability binding assay; preferably such uses, processes, plants or seeds wherein there is no biologically significant competition between the specific binding of any of said Cry2A protein and a Cry1A, Cry1F or VIP3 protein, in standard competition binding assays as described herein, in H. armigera or H. zea.

In the above plants, seeds, uses or methods of the current invention, preferred plants, such as for stacking or combining different chimeric genes in the same plants by crossing, are plants comprising any one of the above corn events or any one of the above cotton events, as well as their progeny or descendants comprising said Cry2A, and said VIP3 and/or Cry1 protein-encoding chimeric genes.

Plants or seeds as used herein include plants or seeds of any plant species significantly damaged by cotton bollworms, but particularly include corn, cotton, rice, soybean, sorghum, tomato, sunflower and sugarcane.

Further provided herein is a method for deregulating or for obtaining regulatory approval for planting or commercialization of plants expressing proteins insecticidal to H. zea or H. armigera, or for obtaining a reduction in structured refuge area containing plants not producing any protein insecticidal to H. zea or H. armigera, or for planting fields without a structured refuge area, such method comprising the step of referring to, submitting or relying on insect assay binding data showing that Cry2A proteins bind specifically and saturably to the insect midgut membrane of such insects, and that said Cry2A proteins do not compete with binding sites for Cry1A, Cry1F or VIP3 proteins in such insects, such as the data disclosed herein or similar data reported in another document. In one embodiment such Cry2A protein is a Cry2Aa, Cry2Ab or Cry2Ae protein and such Cry1A protein is a Cry1Ac, Cry1Ab, or Cry1A.105 protein, and said VIP3 protein is a VIP3Aa protein.

In order to express all or an insecticidally effective part of the DNA sequence encoding a Cry2A, VIP3 or Cry1 protein in E. coli, in other Bt strains and in plants, suitable restriction sites can be introduced, flanking the DNA sequence. This can be done by site-directed mutagenesis, using well-known procedures (Stanssens et al., 1989; White et al., 1989). In order to obtain improved expression in plants, the codon usage of the genes or insecticidally effective gene part of this invention can be modified to form an equivalent, modified or artificial gene or gene part in accordance with PCT publications WO 91/16432 and WO 93/09218 and publications EP 0 385 962, EP 0 359 472 and U.S. Pat. No. 5,689,052, or the genes or gene parts can be inserted in the plastid, mitochondrial or chloroplast genome and expressed there using a suitable promoter (e.g., Mc Bride et al., 1995; U.S. Pat. No. 5,693,507, WO 2004/053133).

Because of the degeneracy of the genetic code, some amino acid codons can be replaced by others without changing the amino acid sequence of the protein. Furthermore, some amino acids can be substituted by other equivalent amino acids without significantly changing, preferably without changing, the insecticidal activity of the protein, at least without changing the insecticidal activity of the protein in a negative way. For example conservative amino acid substitutions within the categories basic (e.g. Arg, His, Lys), acidic (e.g. Asp, Glu), nonpolar (e.g. Ala, Val, Gly, Leu, Ile, Met) or polar (e.g. Ser, Thr, Cys, Asn, Gln) fall within the scope of the invention as long as the insecticidal activity of the protein is not significantly decreased. In addition non-conservative amino acid substitutions fall within the scope of the invention as long as the insecticidal activity of the protein is not significantly decreased. Variants or equivalents of the DNA sequences of the invention include DNA sequences having a different codon usage compared to the native genes of the Cry2A, VIP3, Cry1F or Cry1A proteins used in this invention but which encode a protein with the same insecticidal activity and with substantially the same, preferably the same, amino acid sequence. The DNA sequences can be codon-optimized by adapting the codon usage to that most preferred in plant genes, particularly to genes native to the plant genus or species of interest (Bennetzen & Hall, 1982; Itakura et al., 1977) using available codon usage tables (e.g. more adapted towards expression in corn, cotton, rice, soybean, sorghum, tomato, sunflower or sugarcane). Codon usage tables for various plant species are published for example by Ikemura (1993) and Nakamura et al. (2000).

For obtaining enhanced expression in monocot plants such as corn, sugarcane or rice, an intron, preferably a monocot intron, can also be added to the chimeric gene. For example, the insertion of the intron of the maize Adh1 gene into the 5′ regulatory region has been shown to enhance expression in maize (Callis et. al., 1987). Likewise, the HSP70 intron, as described in U.S. Pat. No. 5,859,347, may be used to enhance expression. The DNA sequence of the insecticidal protein gene or its insecticidal part can be further changed in a translationally neutral manner, to modify possibly inhibiting DNA sequences present in the gene part by means of site-directed intron insertion and/or by introducing changes to the codon usage, e.g., adapting the codon usage to that most preferred by plants, preferably the specific relevant target plant species/genus (Murray et al., 1989), without changing significantly, preferably without changing, the encoded amino acid sequence.

In one embodiment of the invention, cotton bollworms (Helicoverpa zea or Helicoverpa armigera) susceptible to a VIP3, Cry2A, a Cry1F or Cry1A protein are contacted with a combination of these proteins in insect-controlling amounts, preferably insecticidal amounts, e.g., by expressing these proteins in plants targeted by these armyworms or by transforming plants so that these plants and their descendants contain chimeric genes encoding such proteins. In one embodiment target plants for these armyworms are corn, cotton, rice, soybean, sorghum, tomato, sunflower or sugarcane plants, particularly in Northern, Central and Southern American countries. The term plant, as used herein, encompasses whole plants as well as parts of plants, such as leaves, stems, flowers or seeds. In one embodiment at least 3 different proteins of the invention binding to different binding sites in H. zea or H. armigera are combined in such target plants by providing them with the necessary chimeric genes encoding such proteins, such as any one of the following combinations of Cry or VIP3 proteins: a Cry2Ab, a Cry1Ab and a VIP3A protein; a Cry2Ab, a Cry1Ac and a VIP3A protein; a Cry2Ab, a Cry1F and a VIP3A protein; a Cry2Ab, a Cry1A.105 and a VIP3A protein; a Cry2Ae, a Cry1Ab and a VIP3A protein; a Cry2Ae, a Cry1A.105 and a VIP3A protein; a Cry2Ae, a Cry1Ac and a VIP3A protein; or a Cry2Ae, a Cry1F and a VIP3A protein.

The insecticidally effective gene, preferably the chimeric gene, encoding an insecticidally effective portion of the Cry2A, VIP3, Cry1F or Cry1A protein, can be stably inserted in a conventional manner into the nuclear genome of a single plant cell, and the so-transformed plant cell can be used in a conventional manner to produce a transformed plant that is insect-resistant. In this regard, a T-DNA vector, containing the insecticidally effective gene, in Agrobacterium tumefaciens can be used to transform the plant cell, and thereafter, a transformed plant can be regenerated from the transformed plant cell using the procedures described, for example, in EP 0 116 718, EP 0 270 822, PCT publication WO 84/02913 and published European Patent application EP0 242 246 and in Gould et al. (1991). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718. Preferred T-DNA vectors each contain a promoter operably linked to the insecticidally effective gene between T-DNA border sequences, or at least located to the left of the right border sequence. Border sequences are described in Gielen et al. (1984). Of course, other types of vectors can be used to transform the plant cell, using procedures such as direct gene transfer (as described, for example in EP 0 223 247), pollen mediated transformation (as described, for example in EP 0 270 356 and WO 85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods such as the recently described methods for transforming certain lines of corn (e.g., U.S. Pat. No. 6,140,553; Fromm et al., 1990; Gordon-Kamm et al., 1990) and rice (Shimamoto et al., 1989; Datta et al. 1990) and the method for transforming monocots generally (PCT publication WO 92/09696). For cotton transformation, especially preferred is the method described in PCT patent publication WO 00/71733. For rice transformation, reference is made to the methods described in WO92/09696, WO94/00977 and WO95/06722.

The combined expression of a Cry2A and a VIP3, Cry1F or Cry1A protein is most useful in plants targeted by (or damaged by) the cotton bollworm, including corn (field and sweet corn), cotton, tomato, artichoke, asparagus, aubergines, cabbage, cantaloupe, collard, cowpea, cucumber, eggplant, lettuce, lima bean, melon, okra, pepper, potato, pumpkin, snap bean, spinach, squash, sweet potato, watermelon, alfalfa, clover, flax, oat, millet, rice, sorghum, soybean, sugarcane, sunflower, tobacco, vetch, wheat, tobacco, linseed, peas such as pigeonpea or chickpea, chili, okra, besides carnations, geraniums and other ornamental or flower crops, or fruit crops; preferably in corn, cotton, rice, soybean, sunflower, tomato, or sugarcane plants. Hence, the combined use of a Cry2A protein and a VIP3, Cry1F or Cry1A protein in accordance with the invention, for delaying or preventing resistance development of cotton bollworm, is preferably in any one of these plants. The term “corn” is used herein to refer to Zea mays. “Cotton” as used herein refers to Gossypium spp., particularly G. hirsutum and G. barbadense. The term “rice” refers to Oryza spp., particularly O. sativa. “Soybean” refers to Glycine spp, particularly G. max. Sugarcane is used herein to refer to plants of the genus Saccharum, a tall perennial grass of the family Poaceae, native to warm temperate to tropical regions that can be used for sugar extraction. Sunflower as used herein refers to Helianthus annuus.

Transformed plants can be used in a conventional plant breeding scheme to produce more transformed plants with the same characteristics or to introduce the insecticidally effective gene part into other varieties of the same or related plant species. Seeds, which are obtained from the transformed plants, contain the insecticidally effective gene as a stable genomic insert. Cells of the transformed plant can be cultured in a conventional manner to produce the insecticidally effective portion of the Cry2A, VIP3 or Cry1 toxin or protein, which can be recovered for use in conventional insecticide compositions against Lepidoptera.

The insecticidally effective gene is inserted in a plant cell genome so that the inserted gene is downstream (i.e., 3′) of, and under the control of, a promoter which can direct the expression of the gene part in the plant cell (a plant-expressible promoter). This is preferably accomplished by inserting the chimeric gene in the plant cell genome, particularly in the nuclear or plastid (e.g., chloroplast) genome.

Plant-expressible promoters that can be used in the invention include but are not limited to: the strong constitutive 35S promoters (the “35S promoters”) of the cauliflower mosaic virus (CaMV) of isolates CM 1841 (Gardner et al., 1981), CabbB-S (Franck et al., 1980) and CabbB-JI (Hull and Howell, 1987); the 35S promoter described by Odell et al. (1985), promoters from the ubiquitin family (e.g., the maize ubiquitin promoter of Christensen et al., 1992, EP 0 342 926, see also Cornejo et al., 1993), the gos2 promoter (de Pater et al., 1992), the emu promoter (Last et al., 1990), Arabidopsis actin promoters such as the promoter described by An et al. (1996), rice actin promoters such as the promoter described by Zhang et al. (1991) and the promoter described in U.S. Pat. No. 5,641,876; promoters of the Cassava vein mosaic virus (WO 97/48819, Verdaguer et al. (1998)), the pPLEX series of promoters from Subterranean Clover Stunt Virus (WO 96/06932, particularly the S7 promoter), a alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T-DNA (Velten et al., 1984). Alternatively, a promoter can be utilized which is not constitutive but rather is specific for one or more tissues or organs of the plant (e.g., leaves and/or roots) whereby the inserted gene part is expressed only in cells of the specific tissue(s) or organ(s). For example, the insecticidally effective gene could be selectively expressed in the leaves of a plant (e.g., corn, cotton, rice, soybean) by placing the insecticidally effective gene part under the control of a light-inducible promoter such as the promoter of the ribulose-1,5-bisphosphate carboxylase small subunit gene of the plant itself or of another plant, such as pea, as disclosed in U.S. Pat. No. 5,254,799. The promoter can, for example, be chosen so that the gene of the invention is only expressed in those tissues or cells on which the target insect pest feeds so that feeding by the susceptible target insect will result in reduced insect damage to the host plant, compared to plants which do not express the gene. Another alternative is to use a promoter whose expression is inducible, e.g., the MPI promoter described by Cordera et al. (1994), which is induced by wounding (such as caused by insect feeding), or a promoter inducible by a chemical, such as dexamethasone as described by Aoyama and Chua (1997) or a promoter inducible by temperature, such as the heat shock promoter described in U.S. Pat. No. 5,447,858, or a promoter inducible by other external stimuli.

The insecticidally effective gene is inserted into the plant genome so that the inserted gene is upstream (i.e., 5′) of suitable 3′ end transcription regulation signals (i.e., transcript formation and polyadenylation signals). This is preferably accomplished by inserting the chimeric gene in the plant cell genome. The type of polyadenylation and transcript formation signals is not critical, and can include those of the CaMV 35S gene, the nopaline synthase gene (Depicker et al., 1982), the octopine synthase gene (Gielen et al., 1984) or the T-DNA gene 7 (Velten and Schell, 1985), which act as 3′-untranslated DNA sequences in transformed plant cells.

The selection of marker genes for the chimaeric genes of this invention also is not critical, and any conventional DNA sequence can be used which encodes a protein or polypeptide which renders plant cells, expressing the DNA sequence, readily distinguishable from plant cells not expressing the DNA sequence (EP 0344029). The marker gene can be under the control of its own promoter and have its own 3′ non-translated DNA sequence as disclosed above, provided the marker gene is in a genetic locus in the vicinity of the locus of the gene(s) which it identifies. The marker gene can be, for example: a herbicide resistance gene, such as the sfr or sfrv genes (EPA 87400141); a gene encoding a modified target enzyme for a herbicide having a lower affinity for the herbicide than the natural (non-modified) target enzyme, such as a modified 5-EPSP as a target for glyphosate (U.S. Pat. No. 4,535,060; EP 0218571) or a modified glutamine synthetase as a target for a glutamine synthetase inhibitor (EP 0240972); or an antibiotic resistance gene, such as a neo gene (PCT publication WO 84/02913; EP 0193259).

Different conventional procedures can be followed to obtain a combined expression of at least two insecticidal protein genes in transgenic plants, as summarized in EP 408403, incorporated herein by reference. These include transformation of single genes in different plants and crossing such plants, crossing plants already having incorporated each of the desired genes, retransformation of plant already transformed with one gene with the second gene, cotransformation of plants using different plasmids, transformation with at least two genes on one transforming DNA so the genes are inserted at the same locus, using translational fusion genes (see, e.g., Ho et al. (2006)) for transformation, and the like.

The transgenic plant obtained can be used in further plant breeding schemes. The transformed plant can be selfed to obtain a plant which is homozygous for the inserted genes. If the plant is an inbred line, this homozygous plant can be used to produce seeds directly or as a parental line for a hybrid variety. The gene can also be crossed into open pollinated populations or other inbred lines of the same plant using conventional plant breeding approaches.

Since it has now convincingly been shown that Cry2A proteins bind specifically and saturably to the insect midgut of susceptible target insect pests, it is possible to isolate the receptor molecules that is bound specifically by Cry2A proteins by methods known in the art. Hence, included in this invention is also a process for isolating Cry2A protein receptors, particularly the proteins functioning as Cry2Ab receptor, as well as the many uses that can be had of such an isolated receptor protein. Such isolated receptor molecules can be used in screening assays to find proteins binding to a different receptor, to find proteins with improved binding to the receptor, and the like. Also, the DNA sequence of such Cry2A receptor may provide a useful screening tool for screening for alterations in such DNA, which can be an indication of alteration of the protein resulting in resistance development. Such screening can be done quickly using standard DNA detection tools, such as PCR, from insects collected in the field.

Sequence listing:
SEQ ID No. 1: Cry2Ae1 protein
SEQ ID No. 2: Cry2Ab2 protein
SEQ ID No. 3: Cry1Fa1 protein
SEQ ID No. 4: VIP3Aa1 protein
SEQ ID No. 5: VIP3Af1 protein
SEQ ID No. 6: VIP3Aa19 protein
SEQ ID No. 7: VIP3Aa20 protein
SEQ ID No. 8: Cry1Ac1 protein
SEQ ID No. 9: Cry1A.105 protein
SEQ ID No. 10: Cry1Ab1 protein

The following Examples illustrate the invention, and are not provided to limit the invention or the protection sought.

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson et al. (2000) PCR-Basics: From Background to Bench, First Edition, Springer Verlag, Germany.

### EXAMPLES

### Example 1

### 1.1. Materials and Methods

### Toxin Purification and Activation of Toxins.

B. thuringiensis strain HD73 from the Bacillus Genetic Stock Collection (Columbus, Ohio) expressing Cry1Ac was grown in CCY medium (Stewart et al., 1981) at 28.5° C. with continuous shaking and air supplement for 48 hours. The pelleted insoluble fraction was washed twice with 1M NaCl, 10 mM EDTA, and once with 10 mM KCl. Cry1Ac crystals were solubilized in freshly prepared carbonate buffer (50 mM Na2CO3/NaHCO3, 10 mM DTT; pH 10.5) and incubated at room temperature with shaking at 150 rpm for 2.5 h. Insoluble debris was discarded by centrifugation at 25000×g for 10 min at 4° C. The solubilised Cry1Ac protoxin was activated by incubation with trypsin (Sigma T-8642) with a trypsin:protein ratio of 1:10 (w:w) at 37° C. for 2 h. After centrifugation at 25000×g for 10 min at 4° C., the supernatant was dialysed in buffer A (20 mM Tris-HCl, pH 8.65) and filtered prior to anion exchange purification in a MonoQ 5/5 column using an ÄKTA chromatography system (GE Healthcare, UK). A continuous gradient of buffer B (20 mM Tris-HCl, 1M NaCl, pH 8.65) up to 60% was used to elute the Cry1Ac activated toxin. Strain ECE126 from the Bacillus Genetic Stock Collection (Columbus, Ohio) expressing Cry2Aa was grown and activated as Cry1Ac except that solubilization was performed in NEE buffer (50 mM Na2CO3, 5 mM EDTA, 10 mM EGTA; pH 12.1).

Recombinant B. thuringiensis strain BtIPS78/11 expressing Cry2Ab2 was grown in C2 medium (Donovan et al., 1988) containing 6 μg/ml choramphenicol at 28° C. with shaking at 80 rpm for 47 hours. Following two wash steps in phosphate-buffered saline (PBS) (8 mM Na2HPO4, 2 mM KH2PO4, 150 mM NaCl; pH 7.4) to which 250 mM NaCl was added, the cell pellet was solubilized in NEE buffer. Trypsin was added to a final concentration of 0.3 mg/ml out of a freshly prepared 25 mg trypsin/ml and the mixture was incubated at 37° C. for 75 min and centrifuged. The Cry2Ab toxin solution was precipitated with ammonium sulfate and the resulting pellet was dissolved in TEE buffer (50 mM Tris-HCl, 5 mM EDTA, 10 mM EGTA; pH 8.6).

Recombinant B. thuringiensis var. berliner 1715 cry-strain harbouring plasmid pGA32 expressing Cry2Ae was grown in C2 medium containing erythromycin at 20 μg/ml at 28° C. with shaking at 80 rpm for 144 hours. Following two wash steps in PBS plus 250 mM NaCl, the cell pellet was solubilized in alkaline buffer (0.1 M CAPS, 10 mM EGTA, 5 mM EDTA, pH 12.0) and incubated for 1 hour at 37° C. Trypsin was added (at a 3:1 ratio, w/w) out of a freshly prepared 7.5 mg trypsin/ml and the mixture was incubated overnight at 37° C. and centrifuged. The Cry2Ae toxin solution was dialyzed to PBS.

### Bioassays.

Seven different concentrations of each activated Cry protein were tested using 16 neonate larvae of Helicoverpa armigera for each concentration. A volume of 50 μl of the sample dilutions was applied on the surface of artificial diet dispensed in multiwell plates. One larvae was placed in each well. The plates were incubated at 25±2° C., with a relative humidity of 65±5% and a photoperiod of 16:8 (light:dark). Mortality was evaluated after 7 days. Toxicity data were analyzed using the POLO-PC probit analysis program (LeOra Software, Berkeley, Calif.).

### Midgut Isolation and BBMV Preparation.

Last instar larvae of H. armigera (ANGR strain, CSIRO Entomology, Australia) and H. zea (USDA-ARS, MS) were dissected and the midguts were preserved at −80° C. until required. BBMV were prepared by the differential magnesium precipitation method (Wolfersberger et al., 1987), frozen in liquid nitrogen and stored at −80° C. The protein concentration in the BBMV preparations was determined by the method of Bradford (1976) using bovine serum albumin as standard.

### Radiolabeling of Cry Toxins.

Cry1Ac and Cry2Ab toxins were labeled using the chloramine T method. Na125I (0.5 mCi) (PerkinElmer, Boston, Mass.) was added to 25 microgram of Cry toxin in presence of ⅓ v of 18 mM of chloramine T in PBS. After incubation for 45 s, the reaction was stopped by adding ¼ v of 23 mM potassium metabisulfite in H2O. Finally, ¼ v of 1M NaI was added, and the mixture was loaded onto a PD10 desalting column (GE HealthCare, UK) equilibrated with buffer column (20 mM Tris-HCl, 150 mM NaCl, 0.1% BSA). The purity of the labeled protein was checked by analysing the elution fractions by SDS-PAGE with further exposure of the dry gel to an X-ray film. The specific activity of the labeled toxin was calculated based on the input toxin, the radioactivity eluting in the protein peak, and the percentage of radioactivity in the toxin band vs. that in minor bands as reveled by SDS-PAGE (FIG. 1). The estimated specific activities of 125I-Cry1Ac and 125I-Cry2Ab toxins were 3 mCi/mg, and 2.4 mCi/mg, respectively.

Binding Assays with 125I-labeled Cry1Ac and Cry2Ab.

Prior to use, BBMV were centrifuged for 10 min at 16000×g and resuspended in binding buffer (8 mM Na2HPO4, 2 mM KH2PO4, 150 mM NaCl; pH 7.4; 0.1% bovine serum albumin). Saturation experiments were carried out by incubating 20 microgram of BBMV from H. armigera with increasing amount of 125I-Cry2Ab in binding buffer for 1 h at 25° C. After incubation, samples were centrifuged at 16000×g for 10 min and the pellet was washed twice with 500 microliter of cold binding buffer. The radioactivity retained in the pellet was measured in an LKB 1282 Compugamma CS gamma counter. Non-specific binding was determined by adding an excess of unlabeled Cry2Ab (1 micromolar) to the reaction. Specific binding was calculated by subtracting the non-specific binding from the total binding.

To determine the optimal concentration of BBMV to use in competition experiments, increasing amounts of BBMV were incubated with 0.4 nM and 1.2 nM of labeled Cry1Ac and Cry2Ab, respectively, in a final volume of 0.1 ml of binding buffer for 1 h at 25° C. An excess of unlabeled toxin was used to calculate the non-specific binding.

Competition experiments were done by incubating either 20 microgram of BBMV and 1 nM 125I-Cry2Ab, or 5 microgram of BBMV and 0.6 nM 125I-Cry1Ac, for 1 h at 25° C. in the presence of increasing amounts of unlabeled toxins. For quantitative assays, the fraction of labeled toxin bound to BBMV was determined in a gamma counter. Dissociation constants and concentration of binding sites were calculated using the LIGAND program (Munson and Rodbard, 1980). For qualitative assays, pellets were boiled for 10 min in loading buffer (Laemmli, 1970) and run in SDS-PAGE. The labeled toxin retained in the pellet was detected by autoradiography after 1 week exposure.

### 1.2. Results

Toxicity of Activated Cry Proteins to H. armigera.

The toxicity of the Cry1Ac and Cry2Ab preparations was tested. Activated Cry1Ac and Cry2Ab proteins were toxic to H. armigera larvae and the LC50 values are shown in Table 2. Also activated Cry2Ae protein shows significant insecticidal activity to H. armigera.

Specific Binding of 125I-Cry2Ab to H. armigera BBMV.

As a first approach, specific binding of Cry2Ab was tested by incubating BBMV from H. armigera with radiolabeled Cry2Ab (FIG. 1). Although other labeled contaminants were present in the 125I-Cry2Ab preparation, only the band corresponding to Cry2Ab bound to the BBMV. An excess of unlabeled Cry2Ab drastically reduced binding of 125I-Cry2Ab, indicating that most of this binding was specific. In contrast, an excess of unlabeled Cry1Ac did not decrease binding of labeled Cry2Ab, indicating that Cry2Ab binding sites are not recognized by Cry1Ac.

Saturation of 125I-Cry2Ab Binding to H. armigera BBMV.

Binding of Cry2Ab was shown to be saturable by incubating H. armigera BBMV with increasing concentrations of labeled Cry2Ab. In the conditions used (0.2 mg BBMV protein/ml), the curve deviated from linearity starting approximately at 5 nM 125I-Cry2Ab and reached a maximum at approximately 20 nM (FIG. 2).

Competition Experiments with H. armigera BBMV.

To find out the optimal concentration of BBMV for competition binding experiments, a fixed concentration of 125I-Cry2Ab was incubated with increasing concentrations of H. armigera BBMV. As expected, after subtraction of the non-specific binding, an increase in specific binding was observed corresponding to the increase of binding sites (FIG. 3A). A concentration of 0.2 mg/ml of BBMV was chosen to perform competition binding experiments. A similar experiment carried out with 125I-Cry1Ac indicated an optimal BBMV concentration of 0.05 mg/ml for competition experiments (data not shown).

The 125I-Cry2Ab displacement observed with unlabeled Cry2Ab protein in homologous competition assays confirmed that binding of Cry2Ab in this insect is specific and saturable (FIG. 3B). In our experimental conditions, also some non-specific binding was also observed. Competition binding assays using Cry2Aa and Cry2Ae as heterologous competitors showed that these Cry proteins readily competed with 125I-Cry2Ab (FIG. 3B). In contrast, unlabeled Cry1Ac was unable to compete for 125I-Cry2Ab binding in the range of concentrations tested (higher concentrations of Cry1Ac than those shown in FIG. 3B resulted in precipitation of 125I-Cry2Ab). These results indicate that Cry2Ab binding sites are shared by the other two Cry2 toxins, but not by Cry1Ac.

Competition assays were also carried out with 125I-Cry1Ac using Cry1Ac, Cry2Ab, Cry2Aa, and Cry2Ae as competitors (FIG. 3C). None of the Cry2A proteins competed for binding with 125I-Cry1Ac, confirming the occurrence of different binding sites for Cry1Ac and these Cry2A proteins.

Binding parameters, dissociation constant (Kd) and concentration of binding sites (Rt), were calculated for Cry2Ab and Cry1Ac from homologous competition curves (Table 1). In both cases, homologous competition data fit a single-site model equation. As shown in Table 1, Cry2Ab has slightly more specific binding sites than Cry1Ac in H. armigera, but the affinity of Cry2Ab is lower than the affinity of Cry1Ac for their respective binding sites.

Radiolabeling of Cry2Ab and Cry1Ac was done twice independently and all the experiments described above were repeated with a newly prepared second preparation of radiolabeled Cry1Ac and Cry2Ab. Results obtained with the second set of radiolabeled toxins were similar to those described above.

In competition assays using 125I-Cry2Ab, also for the VIP3Aa and Cry1Fa proteins, no competition for the Cry2Ab binding site is seen in BBMVs of H. armigera.

### Specific Binding of Cry2Ab to H. zea BBMV.

To confirm the model of binding sites obtained from experiments in H. armigera, binding assays were also performed in H. zea. In this insect, specific binding was also observed when increasing amounts of BBMV were incubated with 125I-Cry2Ab. The percentage of bound toxin in H. zea was lower than in H. armigera when the same range of BBMV concentration was used (compare FIGS. 4A and 3A).

125I-Cry2Ab was used to perform homologous and heterologous competition assays in H. zea. As in H. armigera, unlabeled Cry2Ab competed for 125I-Cry2Ab binding sites (FIG. 4B). The homologous competition indirectly demonstrated saturable binding of Cry2Ab in this insect, since the curve reflects a limited number of binding sites in the concentration range of unlabeled competitor. The heterologous competition assay showed that Cry1Ac did not compete with 125I-Cry2Ab.

Experiments with 125I-Cry1Ac showed no competition of Cry2Ab for Cry1Ac binding sites (FIG. 4C), demonstrating the existence of different binding sites for Cry2Ab and Cry1Ac also in H. zea.

Similar saturable binding as seen on H. armigera BBMV can be obtained for the Cry2Ab protein in direct saturability assays on BBMV of H. zea. Also in H. zea the Cry2Aa, Cry2Ab and Cry2Ae proteins bind to a common receptor. Also no competition for binding sites is found between the Cry2Ab and any one of the Cry1Ab, Cry1Ac, Cry1Fa or VIP3Aa protein.

### 1.3. Analysis of Results

Saturation binding experiments carried out with Cry2Ab have shown that there are a limited number of specific receptors in the membrane of susceptible insect midgut cells. An analogous saturation assay with labeled Cry1Ac revealed that, with the same concentration of BBMV, saturation of binding sites was achieved using four times less toxin than in the Cry2Ab assay (data not shown). Our results are in contrast with previous results from Cry2Aa saturation binding assays which concluded that this protein binds non-saturably (English et al., 1994, Jurat-Fuentes et al., 2003; EPA biopesticide factsheet 006487 (2002)). There are a number of potential explanations for the observation of a more or less linear curve in those Cry2Aa saturation experiments. First, these authors used labeled Cry2Aa protoxins rather than activated toxins. Moreover, in one study the labeling was performed with denatured Cry2Aa protein, whereas it was not verified that Cry2Aa can withstand a cycle of denaturation and renaturation under the conditions used (English et al., 1994). Secondly, buffers were used at a pH value at which the Cry2A protoxins are not soluble (Staples et al., 2001). In saturation experiments, the low solubility and tendency to aggregate of Cry2A proteins (Perlak et al., 2001) may lead to a linear increase of recovered radioactivity due to precipitation. Thirdly, the concentration range of Cry2Aa protein used in saturation experiments might not have been sufficient to saturate binding sites. The range used was the same as that for Cry1Ac toxin, but due to the lower affinity of Cry2A protein, a higher concentration of Cry2Aa protein may have been required to show saturation of the binding sites. Fourth, the concentration of BBMV used might have been inadequate to distinguish specific binding. High levels of nonspecific binding of Cry2A proteins (to vesicle components and/or vials or filters) might have masked specific binding if the concentration of binding sites was not high enough.

In some studies with ¹²⁵I-Cry2A toxins, the BBMV binding ability assay (i.e. fixed concentration of labeled toxin and increasing amounts of BBMV) has been presented as a ¹²⁵I-Cry2A saturation assay (i.e. fixed BBMV concentration and increasing amounts of labeled toxin) (Karim and Dean, 2000, Karim et al., 2000b, Luo et al., 2007). Saturability of binding sites cannot be determined if the concentration of binding sites is changing along the experiment. Therefore, the classification of Cry2A binding as saturable (Luo et al., 2007) or nonsaturable (Karim and Dean, 2000, Karim et al., 2000b) was based on the wrong type of experiment in these studies. The present study is the first report describing saturable binding of a Cry2 toxin based on the proper saturation experiment.

Homologous competition assays with Cry2Ab in H. armigera and H. zea in the present study confirmed the existence of a limited number of binding sites. Indeed, if Cry2A binding was non-saturable, there would be an essentially infinite number of binding sites available for binding, and competition of the unlabeled toxin with the labeled toxin for these sites would not be practically possible. In the current experiments, a certain percentage of Cry2Ab binding seems to be non-specific. It is very likely though, that most of the radioactivity catalogued as non-specific binding is actually radioactivity coming from precipitated labeled Cry2Ab.

The analysis of binding parameters from homologous competition assays gave values of the dissociation constant (K_{d}) around 35-fold higher for Cry2Ab toxin than for Cry1Ac in both Helicoverpa species analyzed herein, indicating a lower binding affinity of Cry2Ab. However, differences between toxins in terms of concentration of binding sites were much lower (around 3-fold). The K_{d} values for Cry2Ab and Cry1Ac calculated from competition assays were similar to those calculated from saturation experiments in H. armigera (data not shown). Mandal et al. (2007), suggested that the low receptor-binding affinity of Cry2A toxin reported by some authors is attributed to the presence of an extra N-terminal region in this toxin.

To our knowledge, this is the first report on binding competition assays amongst Cry2A proteins. Heterologous competition experiments with labeled Cry2Ab and unlabeled Cry2Aa and Cry2Ae toxins show that these proteins share common binding sites.

In summary, our results clearly show that Cry2A proteins bind saturably and with high affinity to specific sites in H. armigera and H. zea BBMV. In addition, the high-affinity binding sites are shared among Cry2Aa, Cry2Ab and Cry2Ae, but not with Cry1Ac, VIP3 or Cry1F. Also for Cry1Aa, Cry1Ab and Cry1Ac at least one common high-affinity binding site has been reported in all insect species tested. The demonstration that Cry2A proteins have a saturable, high affinity binding site different from that of Cry1A proteins, has important implications for insect resistance management: it offers a biochemical explanation of why cross-resistance between Cry1A and Cry2A proteins is so rare and provides a solid support for the resistance management strategy of combining cry1A and cry2A genes in the same crop to target H. armigera or H. zea.

### Example 2

Several procedures can be envisaged for obtaining the combined expression of at least two insecticidal protein genes, such as the cry2Ae and cry1Ab genes in transgenic plants, such as corn or cotton plants.

A first procedure is based on sequential transformation steps in which a plant, already transformed with a first chimeric gene, is retransformed in order to introduce a second gene. The sequential transformation preferably makes use of two different selectable marker genes, such as the resistance genes for kanamycin and phosphinotricin acetyl transferase (e.g., the well known pat or bar genes), which confers resistance to glufosinate herbicides. The use of both these selectable markers has been described in De Block et al. (1987).

The second procedure is based on the cotransformation of two chimeric genes encoding different insecticidal proteins on different plasmids in a single step. The integration of both genes can be selected by making use of the selectable markers, linked with the respective genes.

Also, separate transfer of two insecticidal protein genes to the nuclear genome of separate plants can be done in independent transformation events, which can subsequently be combined in a single plant through crossing, and plants comprising the different genes can be selected using DNA marker technology.

Corn plants comprising the MIR162 event (WO 2007/142840, USDA APHIS petition for non-regulated status 07-253-01p) are crossed with corn plants containing a chimeric gene comprising the coding region of SEQ ID No. 9 of WO 2002/057664, creating corn plants expressing a VIP3A and a Cry2Ae insect control protein. Alternatively, corn plants containing event Bt11 (USDA APHIS petition for non-regulated status 95-195-01p) or corn plants containing event MON810 (USDA APHIS petition 96-017-01p) are crossed with corn plants containing a chimeric gene comprising the coding region of SEQ ID No. 9 of WO 2002/057664, creating corn plants expressing a Cry1Ab and a Cry2Ae insect control protein.

Cotton plants comprising event EE-GH6 as described in the PCT patent application claiming priority to European patent application number 07075460 or 07075485 (unpublished) are crossed with cotton plants comprising event EE-GH5 described in PCT patent application PCT/EP2008/002667, to obtain cotton plants expressing a Cry2A and Cry1A protein with built-in insect resistance management for H. zea and H. armigera.

Also cotton plants comprising the COT102 event of USDA APHIS petition 03-155-01p (WO 2004/039986) are crossed with the above 2-gene cotton plants, or with cotton plants containing a chimeric gene comprising the coding region of SEQ ID N. 7 of WO 2002/057664 and a chimeric gene comprising the Cry1Ab coding region of SEQ ID No. 2 of U.S. Pat. No. 7,049,491, so that cotton plants expressing the VIP3A, Cry1Ab and Cry2Ae protein are obtained.

Co-expression of at least two insecticidal protein genes in the individual transformants can be evaluated by insect toxicity tests and by biochemical means known in the art. Specific probes allow for the quantitive analysis of the transcript levels; monoclonal antibodies cross-reacting with the respective gene products allow the quantitative analysis of the respective gene products in ELISA tests; and specific DNA probes allow the characterization of the genomic integrations of the transgenes in the transformants.

Of course, besides the above combinations of Cry2A, VIP3 and Cry1 genes for insect resistance management towards cotton bollworms, these plants can also comprise other transgenes, such as genes conferring protection to other Lepidopteran insect species or to insect species from other insect orders, such as Coleopteran or Homopteran insect species, or genes conferring tolerance to herbicides, and the like.

All patents, patent applications, and publications or public disclosures (including publications on internet, and petitions for non-regulated status) referred to or cited herein are incorporated by reference in their entirety to the extent they are not inconsistent with the explicit teachings of this specification. The citation of any document herein does not mean that such document forms part of the common general knowledge in the art.

### CITED REFERENCES

An et al. (1996) Plant J. 10, 107
Adang et al. (1985) Gene 36, 289-300
Aranda et al. (1996) J. Invertebr. Pathol. 68, 203-212
Aoyama and Chua (1997) Plant Journal 11, 605-612
Ballester et al. (1999) Appl. Environm. Microbiol. 65, 1413-1419
Bennetzen & Hall (1982) J. Biol. Chem. 257, 3026-3031
Bradford (1976) Anal Biochem 72, 248-254
Callis et. al. (1987) Genes Developm. 1, 1183-1200
Chalfant (1975) J. Georgia Entomol. Soc. 10, 32-33
Christensen et al. (1992) Plant Mol. Biol. 18, 675-689
Cordera et al. (1994) The Plant Journal 6, 141
Cornejo et al. (1993) Plant Mol. Biol. 23, 567-581
Crickmore et al. (1998) Microbiol. Molecul. Biol. Revs. 62, 807-813
Crickmore et al. (2008) “Bacillus thuringiensis toxin nomenclature”, www.lifesci.sussex.ac.uk/Home/Neil_Crickmore/Bt/
Dankocsik et al. (1990) Mol. Microbiol. 4, 2087-2094
Datta et al. (1990) Bio/Technology 8, 736-740
De Block et al. (1987) EMBO J. 6, 2513-2518
De Pater et al. (1992) Plant J. 2, 834-844
Depicker et al. (1982) J. Molec. Appl. Genetics 1, 561-573
Donovan et al. (1988) J. Biol. Chem. 263:561-567
English et al. (1994) Insect Biochem. Molec. Biol. 24, 1025-1035
EPA biopesticide factsheet 006487 (2002). Bacillus thuringiensis Cry2Ab2 protein and the Genetic Material Necessary for Its Production in Cotton (006487), Fact Sheet (Environmental Protection Agency, USA (www.epa.gov)
EPA experimental use permit factsheet 006499 (2007) Bacillus thuringiensis Vip3Aa19 protein and the genetic material necessary for its production (vector pCOT1) in Event COT102 cotton plants (006499) Experimental Use Permit Factsheet (Environmental Protection Agency, USA, www.epa.gov)
Estela et al. (2004) Appl. Environ. Microbiol. 70: 1378-1384.
Estruch et al. (1996) Proc Natl Acad Sci USA. 93, 5389-94
Fang et al. (2007) Appl. Environm. Microbiol. 73, 956-961
Ferré et al. (1991) Proc. Natl. Acad. Sci. USA 88, 5119-5123
Fiuza et al. (1996) Appl. Environm. Microbiol. 62, 1544-1549
Franck et al. (1980) Cell 21, 285-294
Fromm et al. (1990) Bio/Technology 8, 833-839
Gardner et al. (1981) Nucleic Acids Research 9, 2871-2887
Gielen et al. (1984) EMBO J. 3, 835-845
Gordon-Kamm et al. (1990) The Plant Cell 2, 603-618
Gould et al. (1991) Plant Physiol. 95, 426-434
Hanahan (1983) J. Mol. Biol. 166, 557-580
Henikoff and Henikoff (1992) Proc. Natl. Academy Science 89, 915-919
Hernandez and Ferre (2005) Appl. Environm. Microbiol. 71, 5627-5629.
Ho et al. (2006) Crop Sci 46, 781-789
Hofmann et al. (1988), Proc. Natl. Acad. Sci. U.S.A. 85, 7844-7848
Hull and Howell (1987) Virology 86, 482-493
Ikemura, 1993, In “Plant Molecular Biology Labfax”, Croy, ed., Bios Scientific Publishers Ltd.
Itakura et al. (1977). Science 198, 1056-1063
Jurat-Fuentes et al. (2003) Appl Environ Microbiol. 69(10):5898-906
Karim and Dean (2000). Curr Microbiol. 41(4):276-83
Karim et al. (2000a) Curr Microbiol. 41(3):214-9
Karim et al. (2000b) Pesticide Biochem. Phisiol. 67:198-216
Laemmli et al. (1970) Nature 227, 680-685
Last et al. (1990) Theor. Appl. Genet. 81, 581-588
Lee et al. (2003) Appl. Environm. Microbiol. 69, 4648-4657
Lee et al. (2006) Biochem. Biophys. Res. Comm. 339, 1043-1047
Liao et al. (2002) J. Invertebr. Pathol. 80:55
Luo et al. (1999) Appl. Environm. Microbiol. 65, 457-464
Luo et al. (2007) J Econ Entomol. 100(3):909-15
Mandal et al. (2007) Protein Eng Des Sel. 20(12):599-606
McBride et al. (1995) Bio/Technology 13, 362
Munson & Rodbard (1980) Anal. Biochem. 107, 220-239
Murray et al. (1989) Nucleic Acids Research 17, 477-498
Nakamura et al. (2000) Nucl. Acids Res. 28, 292
Needleman and Wunsch (1970) J. Mol. Biol. 48, 443-453
Nielsen et al. (1996) Int. J. Neur. Sys., 8, 581-599, 1997
Odell et al. (1985) Nature 313, 810-812
Perlak et al. (2001) Plant J. 27(6):489-501
Rang et al. (2004) Curr. Microbiol. 49, 22-27
Robertson & Preisler (1992) CRC Press, Boca Raton, Fla., USA, 127 p
Sayyed et al. (2000). Appl. Environm. Microb. 66, 1509-1516
Shimamoto et al (1989) Nature 338, 274-276
Stanssens et al. (1989) Nucleic Acids Research 12, 4441-4454
Staples et al. (2001) Curr Microbiol. 42(6):388-92
Stewart et al (1981) Biochem. J. 198, 101-106
USDA-APHIS petition for non-regulated status 06-298-01p (2006) for corn event MON 89034, see notice published in the US Federal Register on Dec. 13, 2007 (72 FR 70817-70819; Docket No. APHIS-2007-0030).
Van Rie et al. (1989) Eur. J. Biochem. 186, 239-247
Velten et al. (1984) EMBO J 3, 2723-2730
Velten and Schell (1985), Nucleic Acids Research 13, 6981-6998
Verdaguer et al. (1998), Plant Mol. Biol. 37, 1055-1067
Von Heijne, Gunnar (1986) Nucl. Acids Res. 14:11, 4683-4690
Wabiko et al. (1986) DNA 5, 305-314
Wolfersberger et al. (1987), Comp. Biochem. Physiol. 86, 301-308
White et al. (1989). Trends in Genet. 5, 185-189
Zhang et al. (1991) The Plant Cell 3, 1155-1165

TABLE 1
Binding parameters in H. armigera and H. zea.

| | H. armigera | | H. zea | |
|---|---|---|---|---|
| Toxin | K_{d} ^{a} | Rₜ ^{b} | K_{d} | Rₜ |
| Cry1Ac ^{c} | 0.08 ± 0.02 ^{e} | 0.96 ± 0.10 | 0.17 ± 0.04 | 10 ± 2 |
| Cry2Ab ^{d} | 3.1 ± 0.5 | 2.5 ± 0.3 | 5.9 ± 1.5 | 2.7 ± 0.5 |

^{a} K_{d} values represent the equilibrium dissociation constant and were calculated from the homologous competition assay and are expressed in nanomolar concentrations.
^{b} Rₜ values represent the binding site concentration and are expressed in picomoles per milligram of BBMV protein.
^{c} Values are the mean of two replicates
^{d} Values are the mean of at least four replicates
^{e} Mean ± SEM.

TABLE 2
Toxicity of Cry1Ac and Cry2Ab activated proteins to neonate larvae
of H. armigera (measured after 7 days).

| Protein | LC₅₀ (ng/cm²) | 95% FL^{a} | Slope ± SE |
|---|---|---|---|
| Cry1Ac | 123 | 23-257 | 1.5 ± 0.3 |
| Cry2Ab | 314 | 112-515 | 2.6 ± 0.6 |

^{a}Fiducial limits at the 95% level.

TABLE 3
VIP3 protein list
(www.lifesci.susx.ac.uk/home/Neil_Crickmore/Bt/vip.html)

| | Prior | NCBI | | |
|---|---|---|---|---|
| VIP3 | name | accession | author | Other reference |
| Vip3Aa1 | Vip3Aa | AAC37036 | Estruch et al | PNAS 93, |
| | | | | 5389-5394 |
| Vip3Aa2 | Vip3Ab | AAC37037 | Estruch et al | PNAS 93, |
| | | | | 5389-5394 |
| Vip3Aa3 | Vip3Ac | | Estruch et al | U.S. Pat. No. |
| | | | | 6,137,033 |
| Vip3Aa4 | PS36A | AAR81079 | Feitelson et al | U.S. Pat. No. |
| | Sup | | | 6,656,908 |
| Vip3Aa5 | PS81F | AAR81080 | Feitelson et al | U.S. Pat. No. |
| | Sup | | | 6,656,908 |
| Vip3Aa6 | Jav90 | AAR81081 | Feitelson et al | U.S. Pat. No. |
| | Sup | | | 6,656,908 |
| Vip3Aa7 | Vip83 | AAK95326 | Cai et al | |
| Vip3Aa8 | Vip3A | AAK97481 | Loguercio et al | |
| Vip3Aa9 | VipS | CAA76665 | Selvapandiyan | |
| | | | et al | |
| Vip3Aa10 | Vip3V | AAN60738 | Doss et al | Protein Expr. |
| | | | | Purif. 26, 82-88 |
| Vip3Aa11 | Vip3A | AAR36859 | Liu et al | |
| Vip3Aa12 | Vip3A- | AAM22456 | Wu and Guan | |
| | WB5 | | | |
| Vip3Aa13 | Vip3A | AAL69542 | Chen et al | Sheng Wu Gong |
| | | | | Cheng Xue |
| | | | | Bao 18, |
| | | | | 687-692 |
| Vip3Aa14 | Vip | AAQ12340 | Polumetla et al | |
| Vip3Aa15 | Vip3A | AAP51131 | Wu et al | |
| Vip3Aa16 | Vip3LB | AAW65132 | Mesrati et al | FEMS Micro |
| | | | | Lett 244, |
| | | | | 353-358 |
| Vip3Aa17 | Jav90 | | Feitelson et al | U.S. Pat. No. |
| | | | | 6,603,063 |
| Vip3Aa18 | | AAX49395 | Cai and Xiao | |
| Vip3Aa19- | Vip3ALD | ABB72459 | Liu et al | |
| 2 | | | | |
| Vip3Aa19 | Vip3A-1 | ABG20428 | Syngenta | |
| Vip3Aa20 | Vip3A-2 | ABG20429 | Syngenta | |
| Vip3Aa21 | Vip | ABD84410 | Panbangred | |
| Vip3Aa22 | Vip3A- | AAY41427 | Lu et al | |
| | LS1 | | | |
| Vip3Aa23 | Vip3A- | AAY41428 | Lu et al | |
| | LS8 | | | |
| Vip3Ab1 | Vip3B | AAR40284 | Feitelson et al | U.S. Pat. No. |
| | | | | 6,603,063 |
| Vip3Ab2 | Vip3D | AAY88247 | Feng and Shen | |
| Vip3Ac1 | PS49C | | Narva et al | US 20040128716 |
| Vip3Ad1 | PS158C2 | | Narva et al | US 20040128716 |
| Vip3Ad2 | ISP3B | CAI43276 | Van Rie et al | WO 03/080656 |
| Vip3Ae1 | ISP3C | CAI43277 | Van Rie et al | WO 03/080656 |
| Vip3Af1 | ISP3A | CAI43275 | Van Rie et al | WO 03/080656 |
| Vip3Af2 | Vip3C | | Syngenta | WO 03/075655 |
| Vip3Ag1 | Vip3B | | Syngenta | WO 02/078437 |
| Vip3Ah1 | Vip3S | ABH10614 | Li and Shen | |
| Vip3Ba1 | | AAV70653 | Rang et al | |
| Vip3Bb1 | Vip3Z | | Syngenta | WO 03/075655 |

### FIGURE LEGENDS

FIG. 1. Autoradiography of 125I-Cry2Ab binding to BBMV from H. armigera.

125I-Cry2Ab was incubated with BBMV in the absence or presence of an excess of competitor and the pellet from centrifuging the binding reaction mixture was subjected to SDS-PAGE and exposed to an X-ray film for a week. Lane 1: 1251-Cry2Ab toxin; lane 2: 1251-Cry2Ab incubated with BBMV in absence of competitor; lane 3: homologous competition (excess of unlabeled Cry2Ab); lane 4: heterologous competition with Cry1Ac.

FIG. 2. Saturation of 125I-Cry2Ab specific binding to H. armigera BBMV.

A fixed amount of BBMV (20 microgram of vesicle proteins) was incubated with increasing amounts of 125I-Cry2Ab for 1 h. The binding reaction was stopped by centrifugation and the radioactivity retained in the pellet was measured. Nonspecific binding was calculated by incubating with an excess of unlabeled Cry2Ab, and subtracted from total binding.

FIG. 3. Binding of 125I-Cry2Ab in to H. armigera BBMV.

(A) Specific binding of 125I-Cry2Ab to increasing concentrations of BBMV. Nonspecific binding was calculated in the presence of an excess of unlabeled Cry2Ab. Data points correspond to the mean of five replicates using two independent 125I-Cry2Ab batches. (B) Competition experiments with 125I-Cry2Ab. (C) Competition experiments with 125I-Cry1Ac. In competition assays, each data point represents the mean of at least two independent replicates.

FIG. 4. Binding of 125I-Cry2Ab to H. zea BBMV.

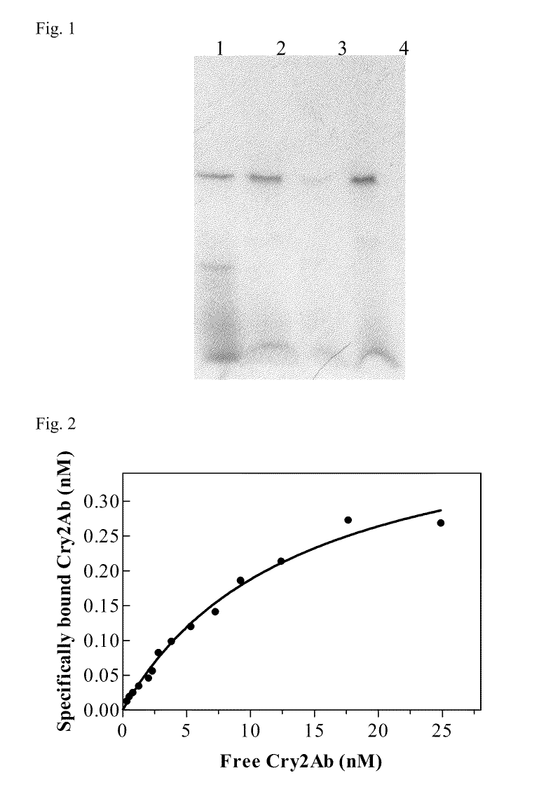

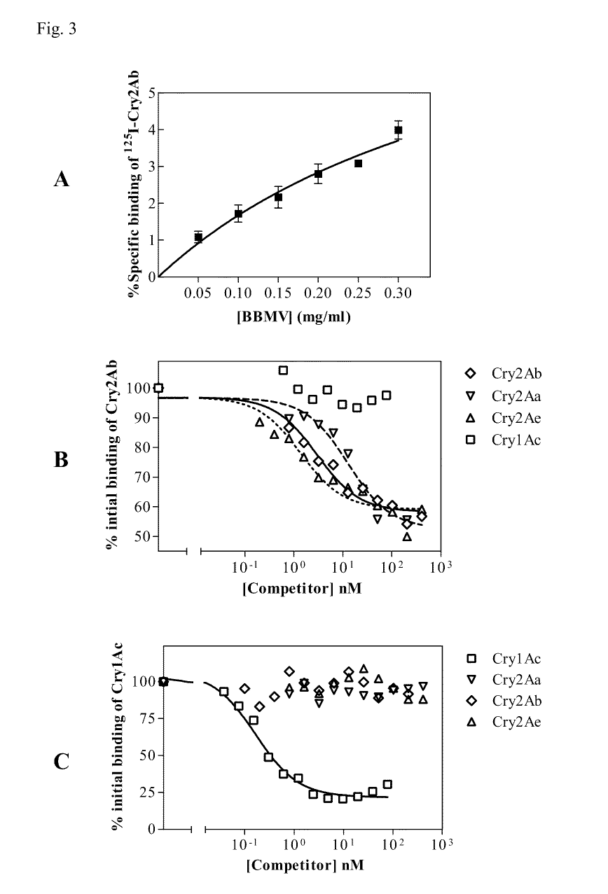

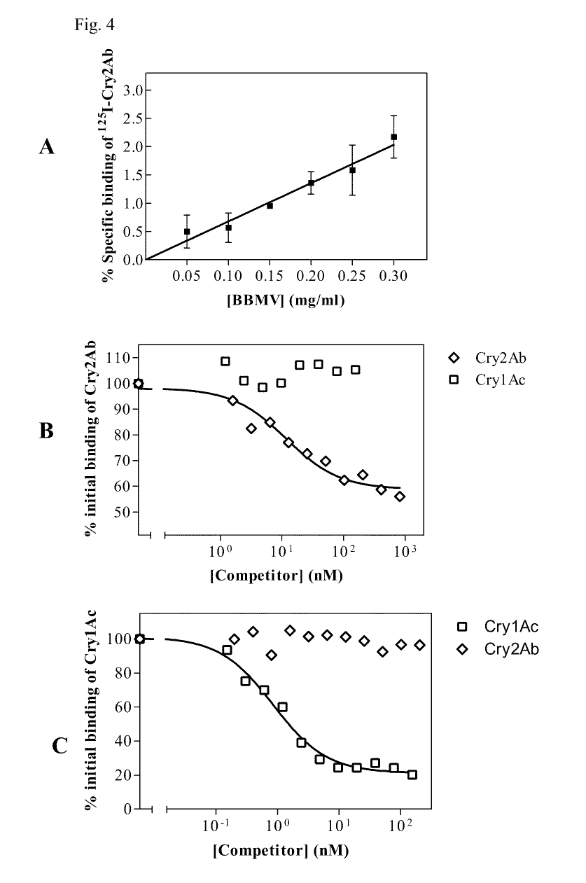

(A) Specific binding of 125I-Cry2Ab to increasing concentrations of BBMV. Nonspecific binding was calculated in the presence of an excess of unlabeled Cry2Ab. (B) Competition experiments with 125I-Cry2Ab. (C) Competition experiments with 125I-Cry1Ac. Each data point represents the mean of at least two independent replicates.

1. A method for preventing or delaying insect resistance development in populations of the insect species Helicoverpa zea or Helicoverpa armigera to transgenic plants expressing insecticidal proteins to control said insect pest, said method comprising expressing a Cry2Ae, Cry2Aa, or Cry2Ab protein insecticidal to Helicoverpa zea or Helicoverpa armigera in combination with a VIP3, Cry1F or Cry1A protein insecticidal to Helicoverpa zea or Helicoverpa armigera in said plants, wherein said insecticidal proteins bind saturable and specific to receptor binding sites in said insects.

2. A method to control Helicoverpa zea or Helicoverpa armigera in a region where populations of said insect have become resistant to plants comprising a VIP3, a Cry1F or a Cry1A protein, comprising the step of sowing or planting in said region, plants comprising a Cry2Aa, Cry2Ab or Cry2Ae protein insecticidal to Helicoverpa zea or Helicoverpa armigera, wherein said insecticidal proteins bind saturable and specific to receptor binding sites in said insects.

3. A method to control Helicoverpa zea or Helicoverpa armigera in a region where populations of said insect have become resistant to plants comprising a Cry2Aa, Cry2Ab or Cry2Ae protein, comprising the step of sowing or planting in said region, plants comprising a VIP3, a Cry1F or a Cry1A protein insecticidal to Helicoverpa zea or Helicoverpa armigera, wherein said insecticidal proteins bind saturable and specific to receptor binding sites in said insects.

4. A method for obtaining plants comprising at least two different insecticidal proteins, wherein said proteins bind saturably and specifically to, and do not share binding sites in, larvae of the species Helicoverpa zea or Helicoverpa armigera as determined in competition binding experiments using brush border membrane vesicles of said insect larvae, comprising the step of obtaining plants comprising a plant-expressible chimeric gene encoding a Cry2Aa, Cry2Ab or Cry2Ae protein insecticidal to Helicoverpa zea or Helicoverpa armigera and a plant-expressible chimeric gene encoding a VIP3, Cry1F or Cry1A protein insecticidal to Helicoverpa zea or Helicoverpa armigera.

5. (canceled)

6. The method of claim 1 to 4, wherein said proteins bind specifically and saturably to, and do not share binding sites in, the larval midgut of said insect species as determined in competition binding experiments using brush border membrane vesicles.

7. The method of any one of claims 1 to 4, wherein said plants comprising a chimeric gene encoding an insecticidal VIP3 protein are plants comprising a chimeric gene encoding a VIP3Aa1, VIP3Aa19, VIP3Aa20, or VIP3Af1 protein, or an insecticidal protein with at least 70% sequence identity to the VIP3A protein of SEQ ID NO. 4, 5, 6 or 7.

8. The method of any one of claims 1 to 4, wherein the plants comprising a Cry1A gene plants comprising a chimeric gene encoding a Cry1Ac, Cry1A.105 or Cry1Ab protein, or an insecticidal protein with at least 90% sequence identity to the Cry1A protein of SEQ ID NO. 8, 9 or 10.

9-10. (canceled)

11. The method of any one of claims 1 to 4, wherein said plants comprising a Cry2Ab gene are plants comprising a chimeric gene encoding a Cry2Ab protein or an insecticidal protein with at least 95% sequence identity to said Cry2Ab protein of SEQ ID No. 2.

12. (canceled)

13. The method of claim 1, wherein said plant is cotton, corn, rice, soybean, tomato, sunflower or sugarcane.

14. The method of claim 1, wherein said method further comprises the planting of a refuge area with plants not comprising a chimeric gene encoding a Cry or VIP protein.

15. The method of claim 1, wherein said plants provide a high dose of Cry2, Cry1 and VIP3 protein for H. zea or H. armigera.

16-19. (canceled)

20. A method for obtaining regulatory approval for planting or commercialization of plants expressing proteins insecticidal to H. zea or H. armigera, comprising the step of referring to, submitting or relying on insect assay binding data showing that Cry2A proteins do not compete with binding sites for Cry1A, Cry1F or VIP3 proteins in such insects.

21. A method for obtaining a reduction in structured refuge area containing plants not producing any protein insecticidal to H. zea or H. armigera in a field, such method comprising the step of referring to, submitting or relying on insect assay binding data showing that Cry2A proteins do not compete with binding sites for Cry1A, Cry1F or VIP3 proteins in such insects.

22. The method of claim 20 or 21, which further comprises the step of referring to, submitting or relying on direct saturability assays showing that Cry2A proteins bind saturably to binding sites in the midgut of H. zea or H. armigera.

23. The method of any one of claims 20 to 22, wherein said Cry2A protein is a protein comprising the smallest toxic fragment of a Cry2Ae protein and wherein said Cry1A, Cry1F or VIP3 protein is any one of the proteins identified in claim 7.

24. A field of insect-resistant transgenic plants controlling H. zea or H. armigera insects, wherein said field has a structured refuge area of less than 20%, of less than 15%, of less than 10%, or of less than 5%, or has no structured refuge area, wherein said plants express a combination of a Cry2Ae or Cry2Ab protein insecticidal to Helicoverpa armigera or Helicoverpa zea insects, and a Cry1A, Cry1F or VIP3A protein insecticidal to Helicoverpa armigera or Helicoverpa zea insects, particularly a Cry2Ae and a Cry1Ab, Cry1Ac or VIP3A protein insecticidal to Helicoverpa armigera or Helicoverpa zea insects, preferably a Cry2Ae, a Cry1Ab and a VIP3 protein, insecticidal to Helicoverpa armigera or Helicoverpa zea insects.


SEQUENCE LISTING (10 sequences)

SEQ ID NO: 1 - protein, 632 aa, Bacillus thuringiensis
MNNVLNNGRTTICDAYNVVAHDPFSFEHKSLDTIRKEWMEWKRTDHSLYVAPIVGTVSSFLLKKVGSLIGKRILSELWGLIFPSGSTNLMQDILRETEQFLNQRLNTDTLARVNAELEGLQANIREFNQQVDNFLNPTQNPVPLSITSSVNTMQQLFLNRLPQFRVQGYQLLLLPLFAQAANMHLSFIRDVVLNADEWGISAATLRTYQNYLKNYTTEYSNYCINTYQTAFRGLNTRLHDMLEFRTYMFLNVFEYVSIWSLFKYQSLLVSSGANLYASGSGPQQTQSFTSQDWPFLYSLFQVNSNYVLNGFSGARLTQTFPNIGGLPGTTTTHALLAARVNYSGGVSSGDIGAVFNQNFSCSTFLPPLLTPFVRSWLDSGSDRGGVNTVTNWQTESFESTLGLRCGAFTARGNSNYFPDYFIRNISGVPLVVRNEDLRRPLHYNEIRNIESPSGTPGGLRAYMVSVHNRKNNIYAVHENGTMIHLAPEDYTGFTISPIHATQVNNQTRTFISEKFGNQGDSLRFEQSNTTARYTLRGNGNSYNLYLRVSSLGNSTIRVTINGRVYTASNVNTTTNNDGVNDNGARFLDINMGNVVASDNTNVPLDINVTFNSGTQFELMNIMFVPTNLPPIY

SEQ ID NO: 2 - protein, 633 aa, Bacillus thuringiensis
MNSVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNISPLY

SEQ ID NO: 3 - protein, 1174 aa, Bacillus thuringiensis
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKPYTRYQLRGYIEDSQDLEIYLIRYNAKHETVNVLGTGSLWPLSVQSPIRKCGEPNRCAPHLEWNPDLDCSCRDGEKCAHHSHHFSLDIDVGCTDLNEDLDVWVIFKIKTQDGHARLGNLEFLEEKPLVGEALARVKRAEKKWRDKREKLELETNIVYKEAKESVDALFVNSQYDQLQADTNIAMIHAADKRVHRIREAYLPELSVIPGVNVDIFEELKGRIFTAFFLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTANQEEYGGAYTSRNRGYDETYGSNSSVPADYASVYEEKSYTDGRRDNPCESNRGYGDYTPLPAGYVTKELEYFPETDKVWIEIGETEGTFIVDSVELLLMEE

SEQ ID NO: 4 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 5 - protein, 788 aa, Bacillus thuringiensis
MNMNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDTDKLFCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGFSQFIGDKLKPKTEYVIQYTVKGKPSIHLKDENTGYIHYEDTNNNLKDYQTITKRFTTGTDLKGVYLILKSQNGDEAWGDKFTILEIKPAEDLLSPELINPNSWITTPGASISGNKLFINLGTNGTFRQSLSLNSYSTYSISFTASGPFNVTVRNSREVLFERSNLMSSTSHISGTFKTESNNTGLYVELSRRSGGGGHISFENVSIK

SEQ ID NO: 6 - protein, 789 aa, Artificial; identical to SEQ ID NO: 4
  designed sequence

SEQ ID NO: 7 - protein, 789 aa, Artificial
  designed sequence
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 8 - protein, 1178 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 9 - protein, 1177 aa, Artificial
  designed sequence
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 10 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE